(19) United States
(12) Patent Application Publication
(10) Pub. No.: US 2024/0218023 A1
(43) Pub. Date: Jul. 4, 2024

(54) CYCLIC PEPTIDE-N-ACETYLGALACTOSAMINE (GALNAC) CONJUGATES FOR DRUG DELIVERY TO LIVER CELLS

(71) Applicant: MICROBIO (SHANGHAI) CO., LTD., Shanghai (CN)

(72) Inventors: Yi-Chung CHANG, Shanghai (CN); Hui-Yu CHEN, Shanghai (CN); Chi-Fan YANG, Shanghai (CN); Huai-Yi CHEN, Shanghai (CN)

(73) Assignee: MICROBIO (SHANGHAI) CO., LTD., Shanghai (CN)

(21) Appl. No.: 18/556,040

(22) PCT Filed: Apr. 21, 2022
(86) PCT No.: PCT/CN2022/088117

§ 371 Date: Oct. 18, 2023

(30) Foreign Application Priority Data
Apr. 23, 2021 (WO) PCT/CN2021/089305

(51) Int. Cl. C07K 9/00; A61K 47/64

(52) U.S. Cl. CPC C07K 9/008; A61K 47/64

(57) ABSTRACT

A conjugate comprising a cyclic peptide scaffold and one or more N-acetylgalactosamine (GalNAc) moieties. The conjugate may further carry a diagnostic or therapeutic agent for use in delivering the agent to liver cells. In some embodiments, the cyclic peptide may have 4-10 amino acid residues. The GalNAc moieties can be covalently bound to the cyclic peptide scaffold via a first linker and the agent can be covalently bound to the cyclic peptide scaffold via a second linker.

### CROSS REFERENCE TO RELATED APPLICATIONS

This application claims the benefit of the filing dates of International Patent Application No. PCT/CN2021/089305, filed on Apr. 23, 2021, the entire contents of which is incorporated by reference herein.

### REFERENCE TO SEQUENCE LISTING SUBMITTED ELECTRONICALLY

The content of the electronically submitted sequence listing in ASCII text file (Name: 112319-0026-70002WO2_SEQ.txt; Size: 2,507 bytes and Date of Creation: Apr. 19, 2022) filed with the application is incorporated herein by reference in its entirety.

### BACKGROUND OF THE INVENTION

N-acetylgalactosamine (GalNAc) has a high binding affinity with asialoglycoprotein receptor (ASGPR), which is highly expressed on hepatocytes. As such, this moiety is commonly used for delivering to liver cells therapeutic or diagnostic agents conjugated to the GalNAc moiety.

GalNAc conjugation is one of the leading approaches for delivering oligonucleotide-based therapeutic agents to liver cells. Multiple GalNAc-siRNA conjugate drug candidates are currently in clinical trials to treat a wide variety of diseases. It is therefore of great interest to develop improved scaffold for preparing GalNAc-drug conjugates having high liver cell targeting efficiency and high endosomal escape efficiency such that the GalNAc-drug conjugates can enter the cytoplasm to induce robust therapeutic effects.

### SUMMARY OF THE INVENTION

The present disclosure is based, at least in part, on the development of cyclic peptide-based scaffold for conjugating GalNAc moieties and agents of interest. The resultant GalNAc-conjugates prepared using such scaffold structures showed high liver cell targeting efficiency and high endosomal escape efficiency. Accordingly, the cyclic peptide-based scaffold and the GalNAc conjugates prepared thereby would be expected to serve as an effective drug delivery platform for targeting liver cells.

Accordingly, in some aspects, the present disclosure provides a conjugate, comprising a cyclic peptide scaffold and one or more N-acetylgalactosamine (GalNAc) moieties. The cyclic peptide scaffold may contain 4-10 amino acid residues. In some embodiments, the cyclic peptide scaffold may contain 4-8 amino acid residues, for example, 4-6 amino acid residues. In one example, the cyclic peptide consists of 6 amino acid residues. In some instances, the cyclic peptide contains Glu, Asp, Lys, Arg, or a combination thereof. For example, the cyclic peptide may contain at least one Glu residue and at least one Lys residue. In addition, the cyclic peptide may further contain Gly, Ala, or Val. The amino acid residues in the cyclic peptide may be in D form.

In some examples, the cyclic peptide scaffold has the amino acid sequence of Lys-Glu-Lys-Gly-Lys-Gly (SEQ ID NO: 5). Alternatively, the cyclic peptide scaffold has the amino acid sequence of Lys-Glu-Lys-Ala-Lys-Ala (SEQ ID NO: 6). One or more amino acid residues in the cyclic peptide scaffold can be in D form. In one example, the cyclic peptide scaffold has the amino acid sequence of Lys-Glu-Lys-βAla-Lys-βAla (SEQ ID NO: 7). Other exemplary cyclic peptide scaffolds include, but are not limited to, CPS-001, CPS-002, CPS-003, and CPS-031. See, e.g., Table 1 and FIGS. 13A-13D). In some instances, the exemplary cyclic peptide scaffold may be a functional equivalent of any one of CPS-001, CPS-002, CPS-003, and CPS-031, which contains the same core structure (e.g., a cyclic peptide scaffold containing the same amino acid residues or an isomer thereof and the same linkers). A functional equivalent of any one of CPS-001, CPS-002, CPS-003, and CPS-031 (the reference conjugate) may be a stereoisomer of a reference conjugate (e.g., S-enantiomer to R-enantiomer switch at one or more chiral centers). Alternatively or on addition, a functional equivalent may contain a different protecting group as the Cbz group in any one of CPS-001, CPS-002, CPS-003, and CPS-031.

In any of the conjugates disclosed herein, each of the GalNAc moieties can be covalently bound to the cyclic peptide scaffold via a first linker. In some instances, the cyclic peptide scaffold includes one or more Lys residues and each first linker can be covalently bound to at least one of the Lys residues. In some examples, each first linker may comprise a linear chain having 3-8 atoms, for example, C, O, or a combination thereof. Specific examples of the first linker can be the linker in Gal-1, Gal-2, Gal-3, Gal-4, or Gal-5. See, e.g., Table 2.

Any of the conjugates disclosed herein may further comprise an agent (e.g., a therapeutic agent or a diagnostic agent), which can be covalently bound to the cyclic peptide scaffold via a second linker. In some instances, the cyclic peptide scaffold includes one or more Glu residues and the second linker can be covalently bound to at least one of the Glu residues. In some examples, the second linker is a lipid linker. In other examples, the second linker can be a polyethylene glycol (PEG) linker. In yet other examples, the second linker can be an alkyl amine linker.

In some examples, the conjugate disclosed herein may have the structure of Formula (I):

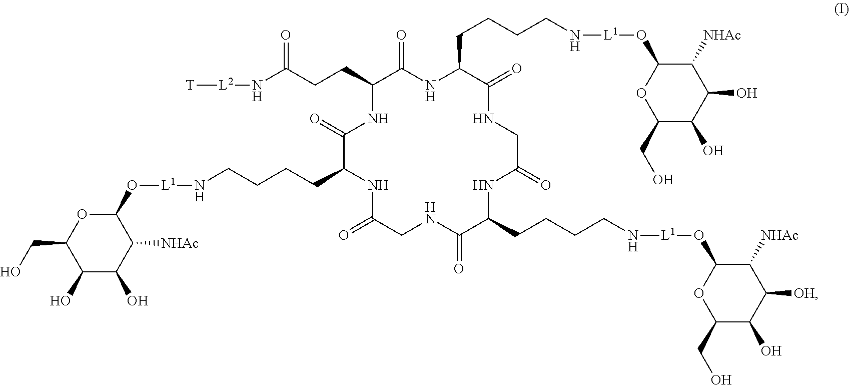

agent; L¹ is the first linker, which can be the linker in Gal-1, Gal-2, Gal-3, Gal-4, or Gal-5; and L² is the second linker.

In other examples, the conjugate disclosed herein can have the structure of Formula (II):

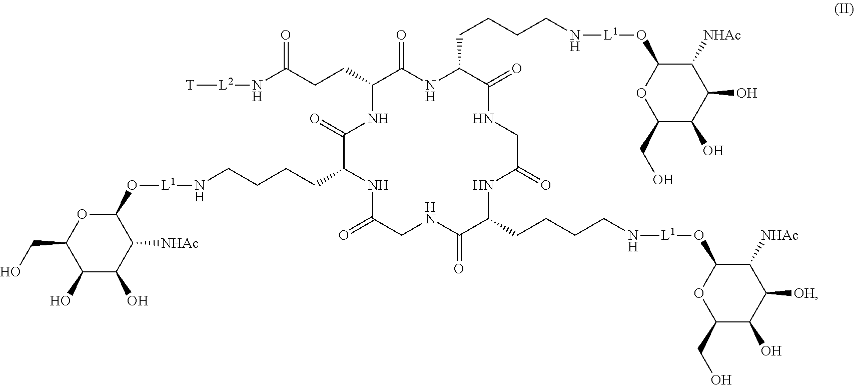

the agent; L¹ is the first linker, which can be the linker in Gal-1, Gal-2, Gal-3, Gal-4, or Gal-5; and L² is the second linker.

In some embodiments, the agent is a diagnostic agent. In other embodiments, the agent can be a therapeutic agent. In some examples, the agent is a small molecule. Alternatively, the agent is a nucleic acid, for example, a small interfering RNA (siRNA), an antisense oligonucleotide (ASO), or a nucleic acid aptamer.

Specific exemplary of the conjugates disclosed herein include 5-FAM-CPMB-0011, 5-FAM-CPMB-0012, 5-FAM-CPMB-0013, 5-FAM-CPMB-0014, 5-FAM-CPMB-0015, 5-FAM-CPMB-0021, 5-FAM-CPMB-0023, 5-FAM-CPMB-0025, 5-FAM-CPMB-0031, 5-FAM-CPMB-0033, 5-FAM-CPMB-0034, 5-FAM-CPMB-0035, 5-FAM-CPMB-0311, 5-FAM-CPMB-0313, CPMB-0013, CPMB-0023, CPMB-0013-DOTMr, or CPMB-0023-DOTMr.

In other aspects, the present disclosure provides a pharmaceutical composition, comprising any of the conjugates disclosed herein and a pharmaceutically acceptable excipient.

Further, provided herein is a method of delivering an agent to liver cells, comprising contacting a liver cell with a conjugate as disclosed herein or a composition comprising such. In some embodiments, the contacting step comprises administering the conjugate or the composition comprising such to a subject in need thereof. In other embodiments, the contacting step comprising incubating the conjugate or the composition comprising such with liver cells in vitro. In this case, the method may further comprise administering to a subject in need thereof the liver cells after being contacted with the conjugate or composition.

Also with the scope of the present disclosure are any of the conjugates or compositions comprising such for use in delivering a diagnostic or therapeutic agent to liver cells, as well as use of such a conjugate or composition comprising such for manufacturing a medicament for use in diagnosing or treating a liver disease.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a schematic diagram illustrating an exemplary synthesis scheme for producing CPMB-002.

FIGS. 2A-2B include schematic diagrams illustrating exemplary synthesis schemes for producing 5-FAM-CPMB-0013. FIG. 2A: exemplary synthesis scheme for producing CPMB-0013-A. FIG. 2B: exemplary synthesis scheme for producing 5-FAM-CPMB-0013 from CPMB-0013-A.

FIG. 3 is a schematic diagram illustrating an exemplary synthesis scheme for producing CPMB-0013.

FIG. 4 is a schematic diagram illustrating an exemplary synthesis scheme for producing CPMB-0013-DMTr.

FIGS. 5A-5B include schematic diagrams illustrating exemplary synthesis schemes for producing CPG-PEG4-CPMB-0013-DMTr. FIG. 5A: exemplary synthesis scheme for producing CPG-PEG4. FIG. 5B: exemplary synthesis scheme for producing CPG-PEG4-CPMB-0013-DMTr from CPG-PEG4.

FIGS. 6A-6B include schematic diagrams illustrating exemplary synthesis schemes for producing 5-FAM-CPMB-0023. FIG. 6A: exemplary synthesis scheme for producing CPMB-0023-A. FIG. 6B: exemplary synthesis scheme for producing 5-FAM-CPMB-0023 from CPMB-0023-A.

FIG. 7 is a schematic diagram illustrating an exemplary synthesis scheme for producing CPMB-0023.

FIG. 8 is a schematic diagram illustrating an exemplary synthesis scheme for producing CPMB-0023-DMTr.

FIG. 9 is a schematic diagram illustrating an exemplary synthesis scheme for producing CPG-PEG4-CPMB-0023-DMTr.

FIG. 10 is a diagram showing improved stability of cyclic peptide-Tri-GalNAc conjugates as compared with tri-GalNAc.

FIG. 11 is a diagram showing endosomal escape of cyclic peptide-Tri-GalNAc conjugates as compared with tri-GalNAc.

FIG. 12 is an illustrative diagram showing the structure of a cyclic peptide-GalNAc conjugate disclosed herein.

FIGS. 13A-13D include diagrams showing structures of representative cyclic peptide scaffolds attached to linkers. FIG. 13A: CPS-001. FIG. 13B: CPS-002. FIG. 13C: CPS-003.

FIG. 13D: CPS-031.

FIGS. 14A-14T include diagrams showing structures of representative cyclic peptide-GalNAc-agent conjugates. FIG. 14A: 5-FAM-CPMB-0011. FIG. 14B: 5-FAM-CPMB-0012.

FIG. 14C: 5-FAM-CPMB-0013. FIG. 14D: 5-FAM-CPMB-0014. FIG. 14E: 5-FAM-CPMB-0015. FIG. 14F: 5-FAM-CPMB-0021. FIG. 14G: 5-FAM-CPMB-0023. FIG. 14H: 5-FAM-CPMB-0025. FIG. 14I: 5-FAM-CPMB-0031. FIG. 14J: 5-FAM-CPMB-0033. FIG. 14K: 5-FAM-CPMB-0034. FIG. 14L: 5-FAM-CPMB-0035. FIG. 14M: 5-FAM-CPMB-0311. FIG. 14N: 5-FAM-CPMB-0313. FIG. 14O: CPMB-0013. FIG. 14P: CPMB-0023. FIG. 14Q: 5-FAM-tri-GalNAc (positive control). FIG. 14R: 5-FAM-CPMB-0031-Ac (negative control). FIG. 14S: CPMB-0013-DOTMr. FIG. 14T: CPMB-0023-DOTMr.

### DETAILED DESCRIPTION OF THE INVENTION

Provided herein are development of cyclic peptide molecules that can serve as scaffold for conjugating multiple copies of N-acetylgalactosamine (GalNAc) moieties (e.g., three) and an agent of interest to be delivered to liver cells (e.g., a therapeutic agent or a diagnostic agent). The GalNAc moieties and/or the agent of interest can be conjugated (e.g., covalently) to the cyclic peptide scaffold via flexible linkers. The flexible linkers can be designed to minimize interference among the various GalNAc moieties and the agent of interest, and to maximize binding affinity of the GalNAc moieties to the ASGPR on liver cells. Exemplary cyclic peptide-GalNAc conjugates provided herein showed high binding activity to liver cells and high endosomal escape rates, indicating that such cyclic peptide-GalNAc conjugates can effectively deliver agents of interest (e.g., nucleic acid-based such as small interfering RNAs, antisense oligonucleotides, or nucleic acid aptamers) inside liver cells to exert the intended bioactivity.

Accordingly, provided herein are cyclic peptide-GalNAc conjugates, pharmaceutical compositions comprising such, and method of using such conjugates to deliver diagnostic or therapeutic agents into liver cells.

### I. Cyclic Peptide Scaffold-GalNAc Conjugates

In some aspects, the present disclosure provides cyclic peptide-GalNAc conjugates, each of which comprises a cyclic peptide scaffold to which one or more GalNAc moieties (e.g., 3) via a flexible linker (the first linker). The conjugates may further comprise an agent of interest via a flexible linker (the second linker).

Some of the compounds according to the present disclosure may exist as stereoisomers, i.e., having the same atomic connectivity of covalently bonded atoms yet differing in the spatial orientation of the atoms. For example, compounds may be optical stereoisomers, which contain one or more chiral centers, and therefore, may exist in two or more stereoisomeric forms (e.g., enantiomers or diastereomers). Thus, such compounds may be present as single stereoisomers (i.e., essentially free of other stereoisomers), racemates, and/or mixtures of enantiomers and/or diastereomers. As another example, stereoisomers include geometric isomers, such as cis- or trans-orientation of substituents on adjacent carbons of a double bond. Unless specified to the contrary, all such stereoisomeric forms are included within the formulae provided herein.

An enantiomer can be characterized by the absolute configuration of its asymmetric center and is described by the (R) and (S) sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Unless otherwise indicated, the description is intended to include individual stereoisomers as well as mixtures. The methods for the determination of stereochemistry and the separation of stereoisomers are well-known in the art (see discussion in Chapter 4 of ADVANCED ORGANIC CHEMISTRY, 6^{h} edition J. March, John Wiley and Sons, New York, 2007) differ in the chirality of one or more stereocenters. All such single stereoisomers, racemates and mixtures thereof are intended to be within the scope of the present disclosure.

### A. Cyclic Peptide Scaffold

The cyclic peptide scaffold used in any of the conjugates disclosed herein may contain 4-10 amino acid residues. In some instances, it may contain 4-8 amino acid residues. In one example, the cyclic peptide scaffold disclosed herein contains 6 amino acid residues.

The cyclic peptide scaffold disclosed herein may contain one or more amino acid residues, the side chain of which contains a functional group (e.g., —COOH, —NH₂, —SH, or —OH). Such a functional group can be used in a chemical reaction for covalent conjugation of a GalNAc moiety (e.g., via a linker) and/or an agent of interest (e.g., via a linker). For example, a cyclic peptide scaffold disclosed herein may contain at least one Arg or Lys (e.g., Lys) residues, the —NH₂ functional group in the side chain of which can be used for covalent conjugation of a GalNAc moiety or an agent of interest. In another example, a cyclic peptide scaffold disclosed herein may contain at least one Asp or Glu residues, the —COOH functional group in the side chain of which can be used for covalent conjugation of a GalNAc moiety or an agent of interest.

In some embodiments, the cyclic peptide scaffold may contain at least two different types of amino acid residues having different functional groups in side chains (e.g., a Lys residue and an Glu residue) to facilitate conjugation of the GalNAc moieties and the agent of interest. For example, the cyclic peptide scaffold may contain multiple Lys residues (e.g., 3 Lys residues), each of which can serve as an anchor for conjugating a GalNAc moiety, and one Asp or Glu amino acid residue, which can serve as the anchor for conjugating the agent of interest.

Any of the cyclic peptide scaffolds disclosed herein may further contain one or more amino acid residues having aliphatic side chains, for example, Gly, Ala, Val, Ile, or Leu. In some examples, the cyclic peptide scaffold may contain Gly, Ala, or a combination thereof.

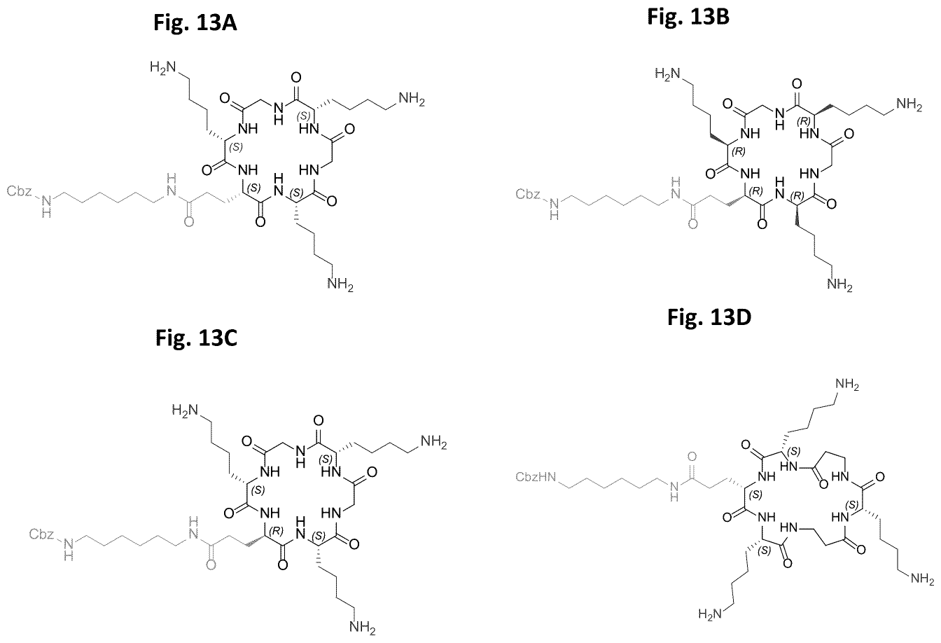

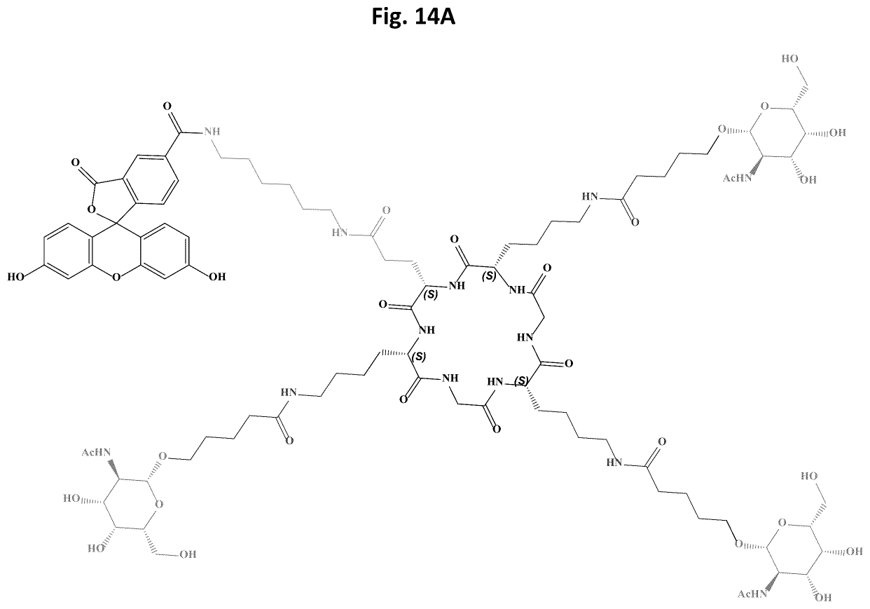

The amino acid residues in the cyclic peptide scaffold may be in L form, in D form, or a mixture thereof. In some examples, the cyclic peptide scaffold may contain at least one amino acid residue in D form, for example, one or more D-Lys or one D-Glu. Exemplary cyclic peptide scaffolds are provided in Table 1 below. See also FIGS. 13A-13D for their chemical structures.

TABLE 1
Exemplary Cyclic Peptide Scaffolds

| Peptide | | Chemical |
|---|---|---|
| ID | Peptide sequence | structure |
| CPS-001 | Cyclic[Lys-Glu(Cbz-Linker2)- | FIG. 13A |
| | Lys-Gly-Lys-Gly] | |
| | (SEQ ID NO: 1) | |
| CPS-002 | Cyclic[DLys-DGlu(Cbz-Linker2)- | FIG. 13B |
| | DLys-Gly-DLys-Gly] | |
| | (SEQ ID NO: 2) | |
| CPS-003 | Cyclic[Lys-DGlu(Cbz-Linker2)- | FIG. 13C |
| | Lys-Gly-Lys-Gly] | |
| | (SEQ ID NO: 3) | |
| CPS-031 | Cyclic[Lys-Glu(Linker2)-Lys-β- | FIG. 13D |
| | -Ala-Lys-β-Ala] | |
| | (SEQ ID NO: 4) | |

### B. GalNAc-Linker Moieties

The conjugates disclosed herein comprises any of the cyclic peptide scaffolds disclosed herein and one or more GalNAc moieties, which can be covalently conjugated to the cyclic peptide scaffold via a flexible linker (first linker).

Any flexible linker may be used in making the conjugates disclosed herein. In some embodiments, the first linker can be a linear chain containing 3-8 atoms, which may be C, O, N, or a combination thereof. Such a length of the first linker could minimize interference among the multiple GalNAc moieties and achieve overall binding affinity to liver cells.

Table 2 below provides exemplary GalNAc-linker structures for use in making the conjugates disclosed herein. The —COOH functional group in Gal-1 to Gal-5 listed in Table 2 can be used to react with an —NH₂ functional group in a cyclic peptide scaffold, leading to covalent conjugation of the GalNAc linker moieties onto the cyclic peptide scaffold.

TABLE 2
Exemplary GalNAc-Linker Structures

| Compound ID | Chemical structure | Linker Structure |
|---|---|---|
| Gal-1 | 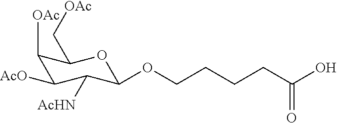 [SMILES: CC(=O)NC1C(OCCCCC(=O)O)OC(COC(C)=O)C(OC(C)=O)C1OC(C)=O] | 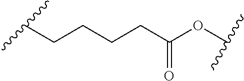 [SMILES: *CCCCC(=O)O*] |
| Gal-2 | 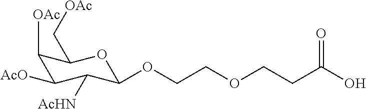 [SMILES: CC(=O)NC1C(OCCOCCC(=O)O)OC(COC(C)=O)C(OC(C)=O)C1OC(C)=O] | 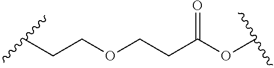 [SMILES: *CCOCCC(=O)O*] |
| Gal-3 | 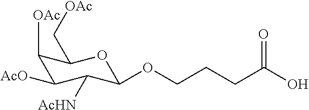 [SMILES: CC(=O)NC1C(OCCCC(=O)O)OC(COC(C)=O)C(OC(C)=O)C1OC(C)=O] | 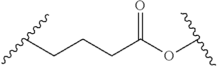 [SMILES: *CCCC(=O)O*] |
| Gal-4 | 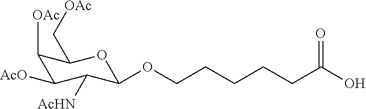 [SMILES: CC(=O)NC1C(OCCCCCC(=O)O)OC(COC(C)=O)C(OC(C)=O)C1OC(C)=O] | 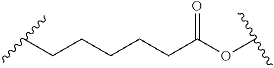 [SMILES: *CCCCCC(=O)O*] |
| Gal-5 | 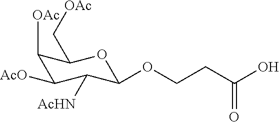 [SMILES: CC(=O)NC1C(OCCC(=O)O)OC(COC(C)=O)C(OC(C)=O)C1OC(C)=O] | 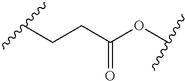 [SMILES: *CCC(=O)O*] |

### C. Agents of Interest

The conjugates disclosed herein may further comprise an agent of interest, which can be conjugated (e.g., covalently) to the cyclic peptide scaffold via a second flexible linker. In some embodiments, the second flexible linker is different from the first flexible linker.

Any flexible linker may be used for conjugating the agent of interest to the cyclic peptide scaffold disclosed herein. Examples include, but are not limited to, lipid linkers, polyethylene glycol linkers, or aliphatic chain linkers. The second flexible linker may contain two functional groups (e.g., two different functional groups) at both ends, one for reacting with the agent of interest, and the other for reacting with the cyclic peptide scaffold. For example, the second linker may contain an —NH₂ functional group at one end, which can reach with an —COOH functional group in the cyclic peptide scaffold (e.g., in an Asp or Glu residue). Selection of the functional group for linking the agent of interest would be determined by the type of agents, e.g., functional groups contained therein, which would be within the knowledge of a skilled person in the pertinent art.

The agent may be of any type, for example, a small molecule, a peptide or polypeptide, an oligosaccharide, a lipid, or a nucleic acid (e.g., RNA or DNA, double strand or single strand). In some embodiments, the agent of interest can be a therapeutic agent, for example, a therapeutic agent for treatment of a liver disease. In other embodiments, the agent of interest can be a diagnostic agent, which may be further conjugated with a label capable of releasing a detectable signal, either directly or indirectly.

In some embodiments, the agent of interest conjugated to the cyclic peptide scaffold can be a nucleic acid, for example, a small interfering RNA, an antisense oligonucleotide (RNA or DNA), a messenger RNA, or a nucleic acid-based aptamer. Any of such nucleic acid may contain non-naturally-occurring nucleobases, sugars, or covalent internucleoside linkages (backbones). Such a modified oligonucleotide confers desirable properties, for example, enhanced cellular uptake, improved affinity to the target nucleic acid, and increased in vivo stability.

In one example, the nucleic acid-based agent described herein (e.g., siRNAs) may have a modified backbone, including those that retain a phosphorus atom (see, e.g., U.S. Pat. Nos. 3,687,808; 4,469,863; 5,321,131; 5,399,676; and 5,625,050) and those that do not have a phosphorus atom (see, e.g., U.S. Pat. Nos. 5,034,506; 5,166,315; and 5,792,608). Examples of phosphorus-containing modified backbones include, but are not limited to, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having 3′-5′ linkages, or 2′-5′ linkages. Such backbones also include those having inverted polarity, i.e., 3′ to 3′, 5′ to 5′ or 2′ to 2′ linkage. Modified backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In another example, the nucleic acid-based agent described herein (e.g., siRNAs) may include one or more substituted sugar moieties. Such substituted sugar moieties can include one of the following groups at their 2′ position: OH; F; O-alkyl, S-alkyl, N-alkyl, O-alkenyl, S-alkenyl, N-alkenyl; O-alkynyl, S-alkynyl, N-alkynyl, and O-alkyl-O-alkyl. In these groups, the alkyl, alkenyl and alkynyl can be substituted or unsubstituted C1 to C10 alkyl or C2 to C10 alkenyl and alkynyl. They may also include at their 2′ position heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide. Preferred substituted sugar moieties include those having 2′-methoxyethoxy, 2′-dimethylaminooxyethoxy, and 2′-dimethylaminoethoxyethoxy. See Martin et al., Helv. Chim. Acta, 1995, 78, 486-504.

Alternatively or in addition, the nucleic acid-based agent described herein (e.g., siRNAs) may include one or more modified native nucleobases (i.e., adenine, guanine, thymine, cytosine and uracil). Modified nucleobases include those described in U.S. Pat. No. 3,687,808, The Concise Encyclopedia Of Polymer Science And Engineering, pages 858-859, Kroschwitz, J. I., ed. John Wiley & Sons, 1990, Englisch et al., Angewandte Chemie, International Edition, 1991, 30, 613, and Sanghvi, Y. S., Chapter 15, Antisense Research and Applications, pages 289-302, CRC Press, 1993. Certain of these nucleobases are particularly useful for increasing the binding affinity of the interfering RNA molecules to their targeting sites. These include 5-substituted pyrimidines, 6-azapyrimidines and N-2, N-6 and 0-6 substituted purines (e.g., 2-aminopropyl-adenine, 5-propynyluracil and 5-propynylcytosine). See Sanghvi, et al., eds., Antisense Research and Applications, CRC Press, Boca Raton, 1993, pp. 276-278).

Alternatively or in addition, the nucleic acid-based agent described herein (e.g., siRNAs) may comprise one or more locked nucleic acids (LNAs). An LNA, often referred to as inaccessible RNA, is a modified RNA nucleotide, in which the ribose moiety is modified with an extra bridge connecting the 2′ oxygen and 4′ carbon. This bridge “locks” the ribose in the 3′-endo (North) conformation, which is often found in the A-form duplexes.

In some examples, the agent may be an intermediate for further attachment or synthesis of a nucleic acid-based therapeutic or diagnostic agent. For example, the agent conjugated to the cyclic peptide scaffold may be a solid support (e.g., control pore glass or CPG), which can be conjugated to the cyclic peptide scaffold via a second linker. The second linker may be attached to the cyclic peptide scaffold via a ribose moiety, which can carry a DMTO protection moiety. This conjugate can be used for adding a desired nucleic acid agent using a nucleic acid synthesis device, which adds nucleotide residues to the ribose moiety via routine nucleic acid synthesis. After synthesis of the nucleic acid agent, which is covalently conjugated to the cyclic peptide scaffold, the final cyclic peptide-GalNAc-nucleic acid conjugates can be released from the solid support (e.g., CPG). See Examples below.

### D. Exemplary GalNAc-Cyclic Peptide Conjugates

The GalNAc-cyclic peptide conjugates disclosed herein may contain any of the cyclic peptide scaffold disclosed herein and one or more GalNAc moieties (e.g., 3) via any of the first linkers disclosed herein. The conjugate may further comprise a second linker, e.g., those disclosed herein, to which any of the agents of interest disclosed herein are attached (e.g., covalently).

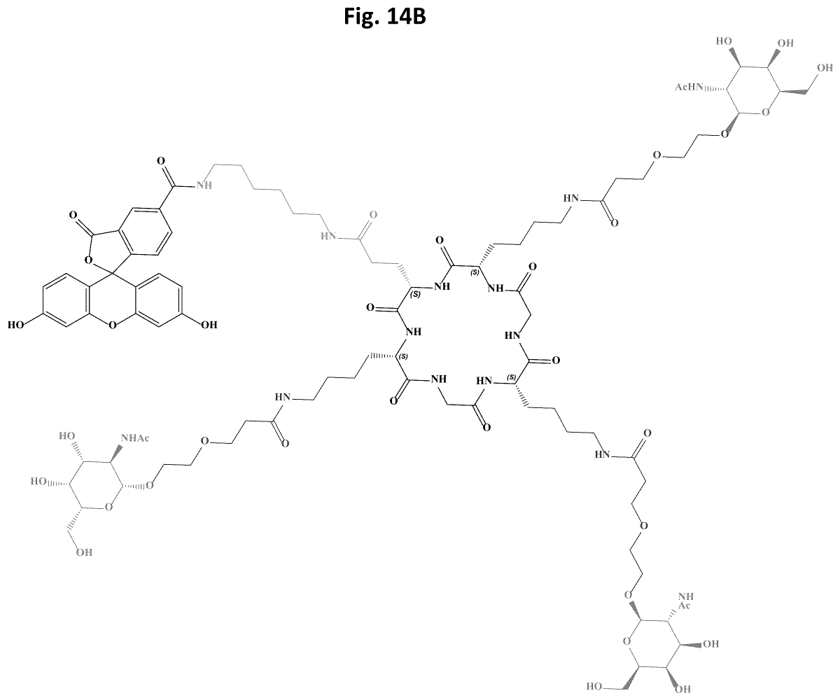

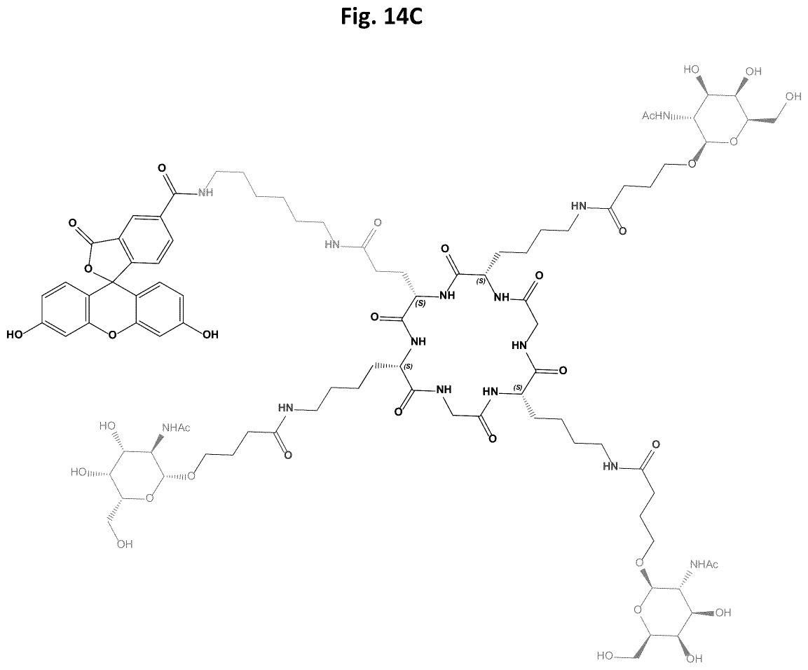

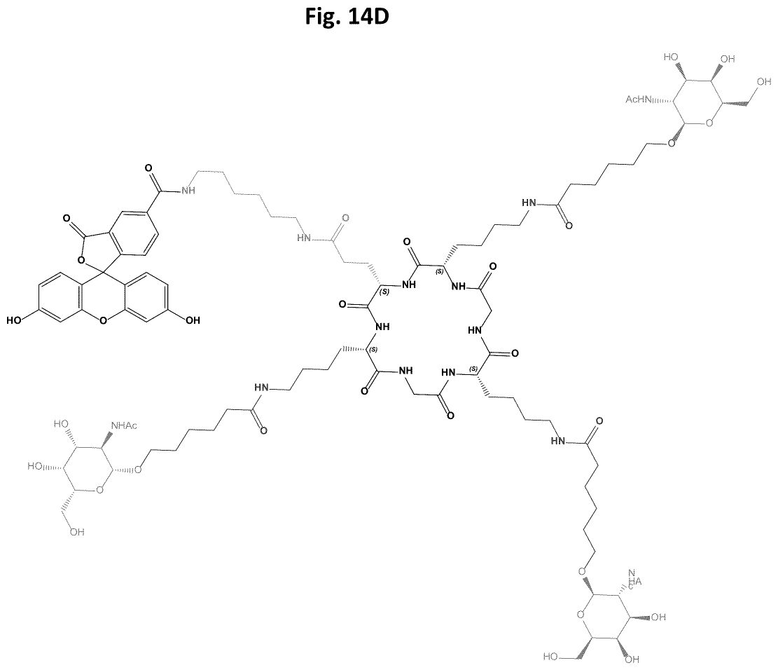

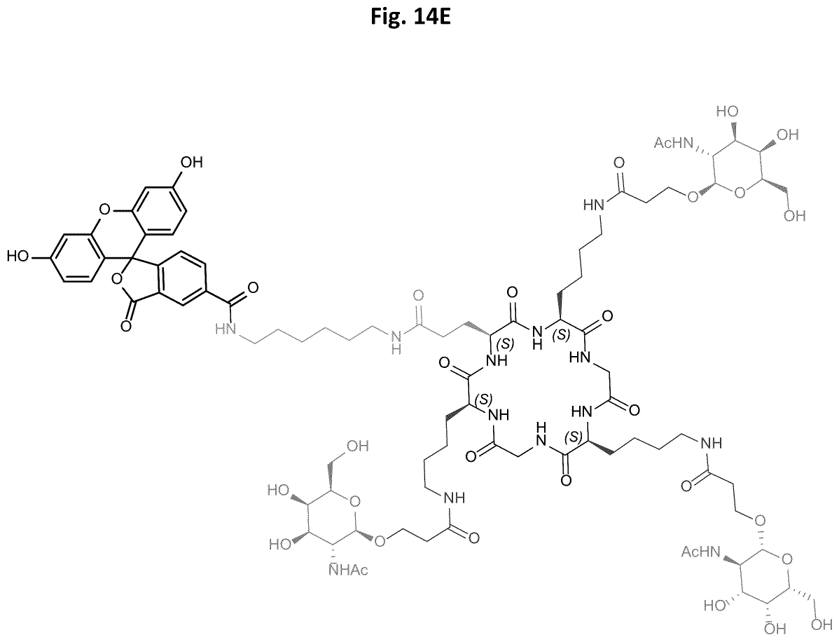

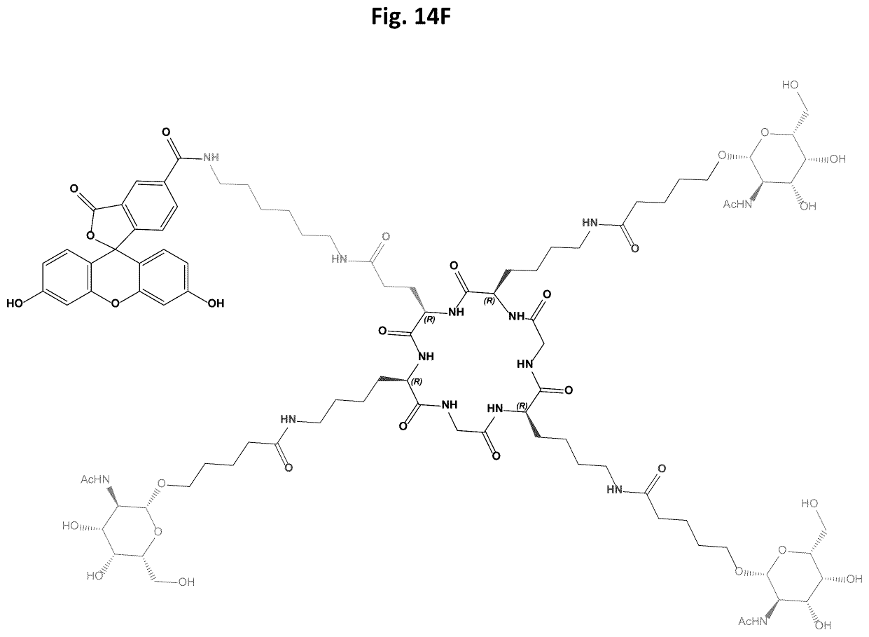

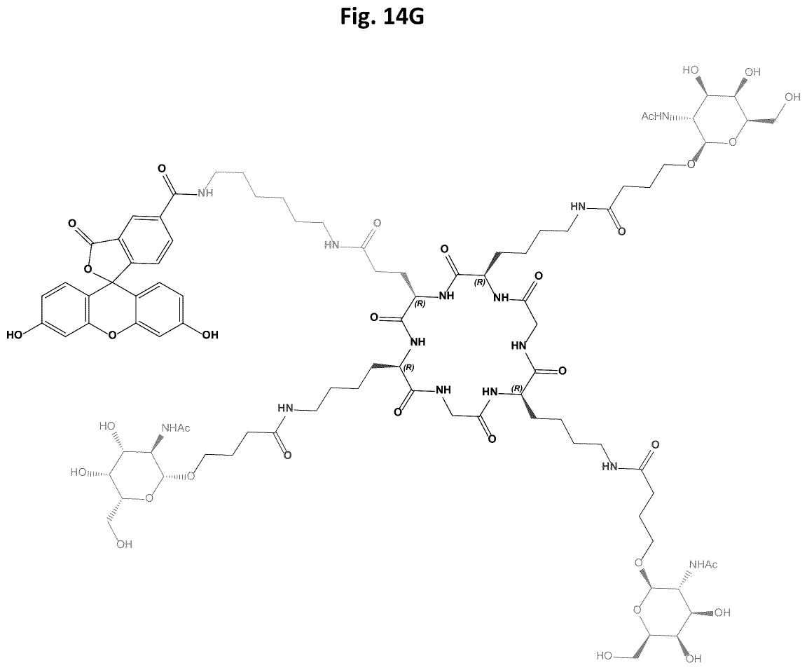

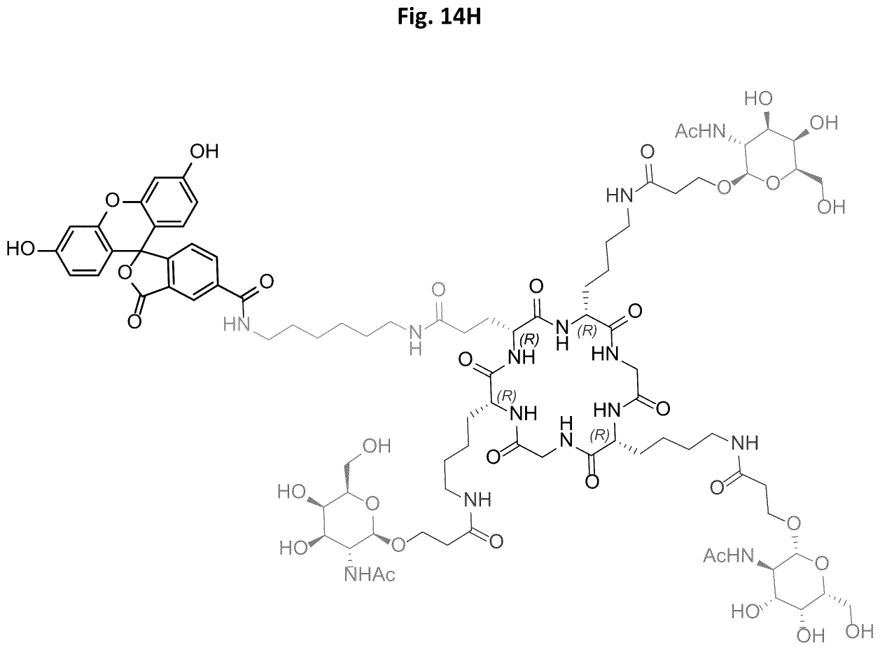

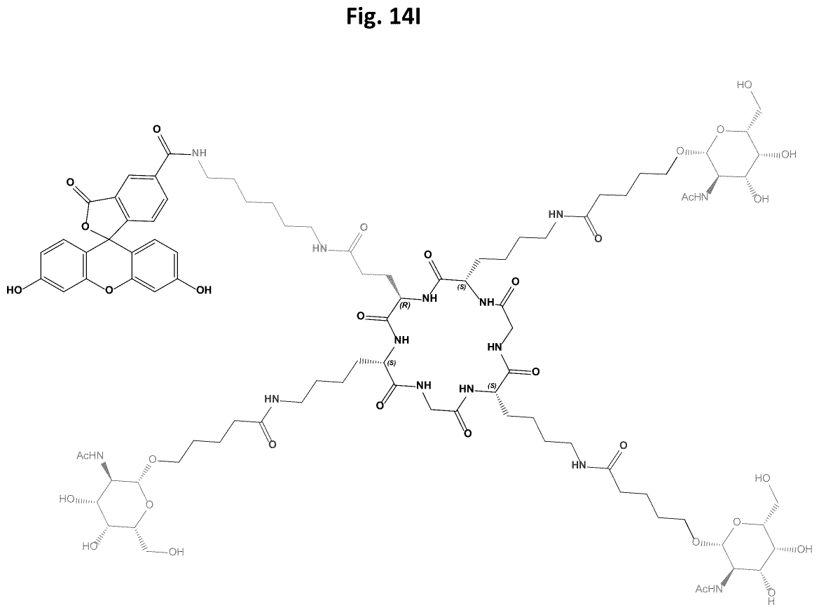

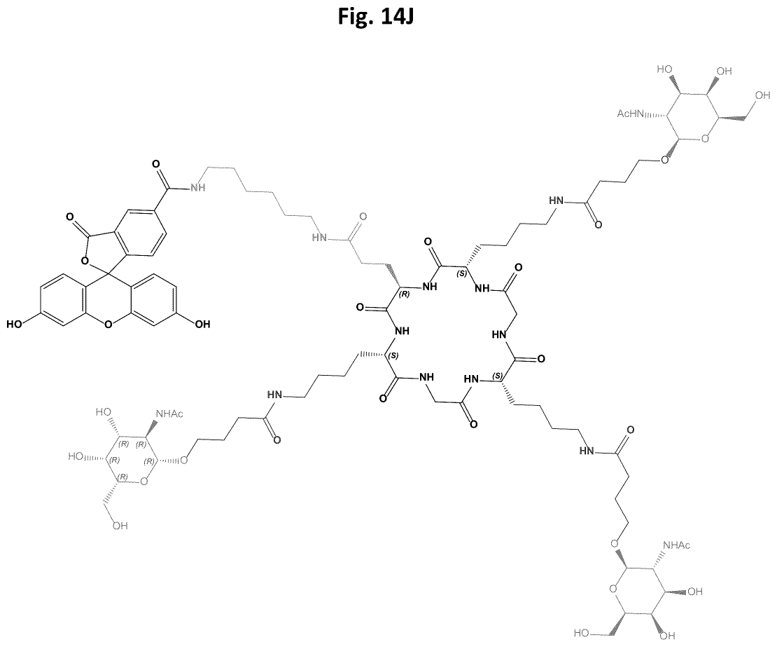

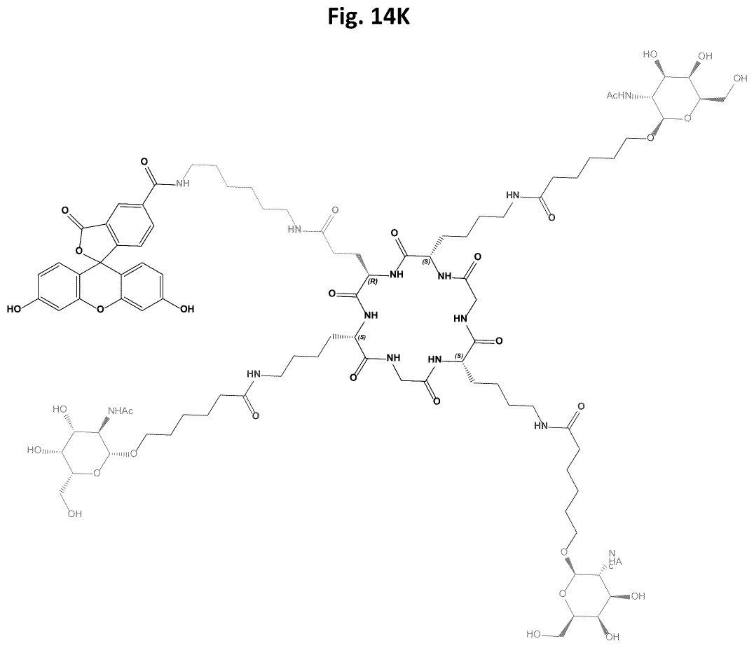

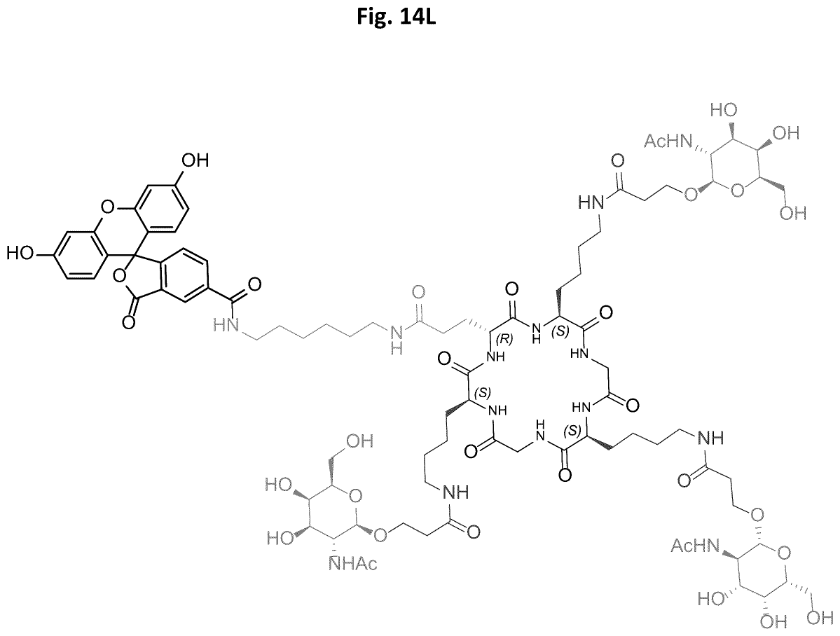

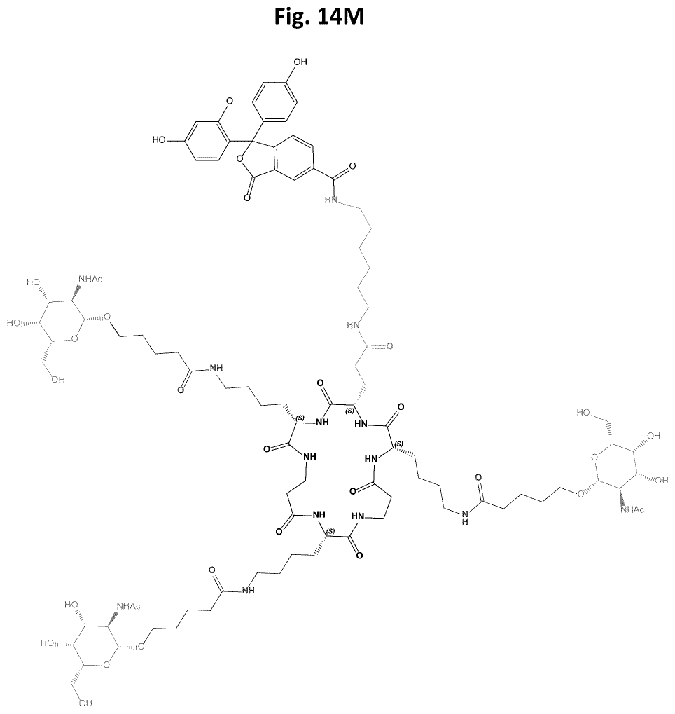

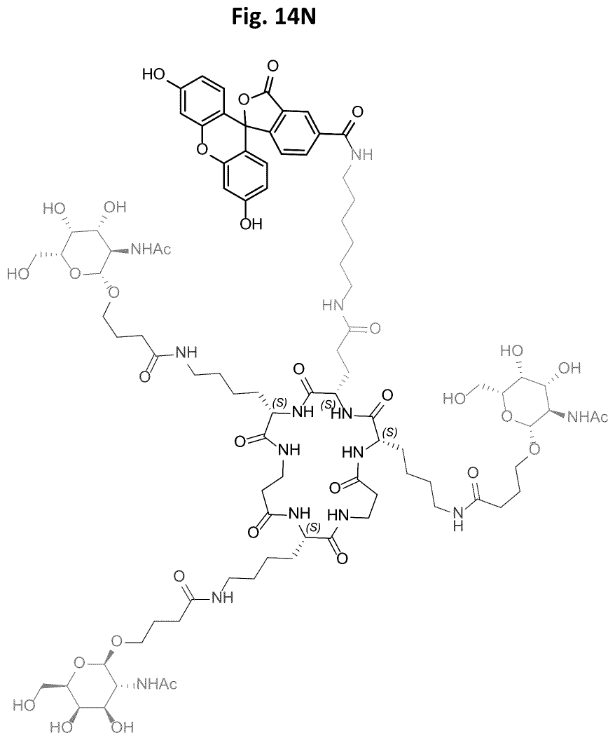

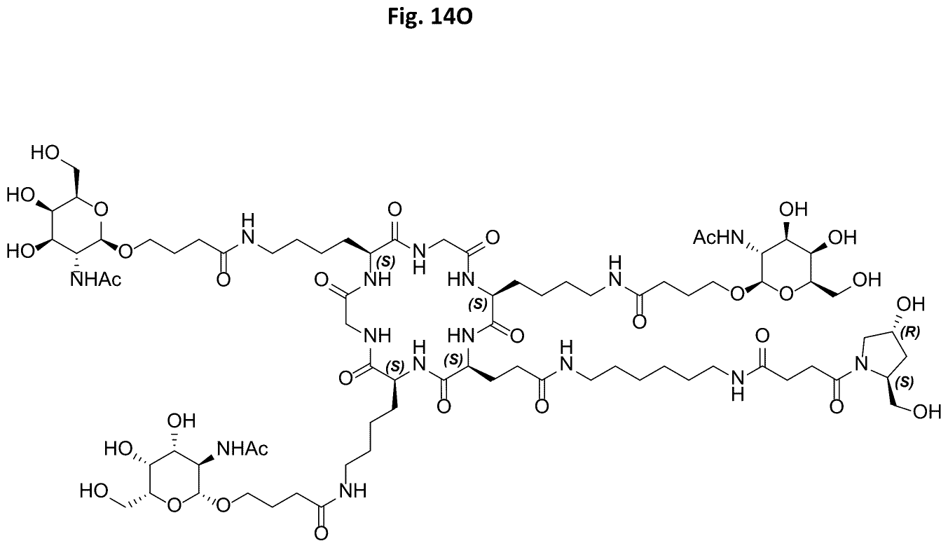

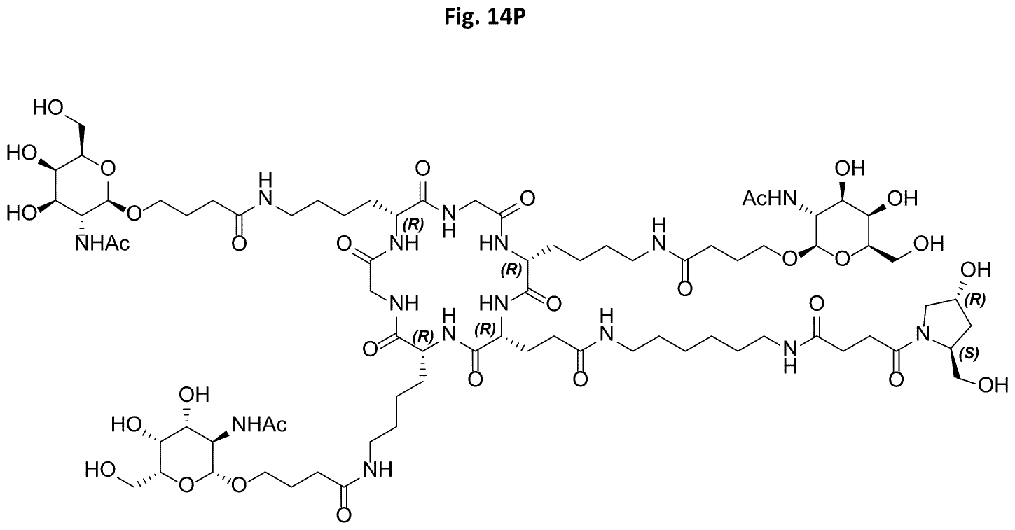

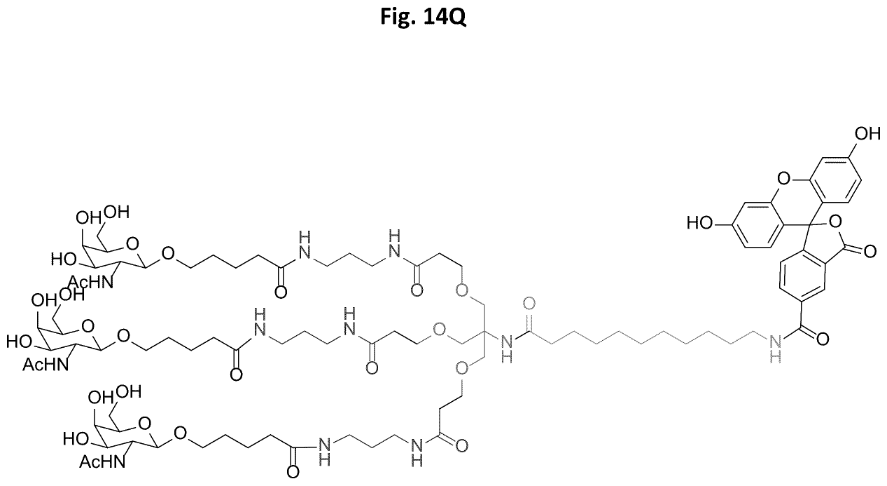

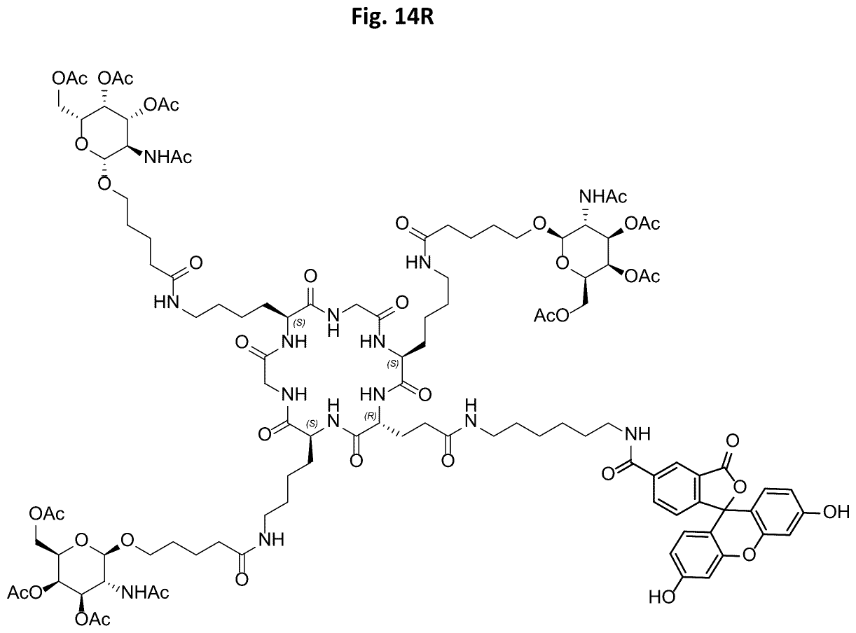

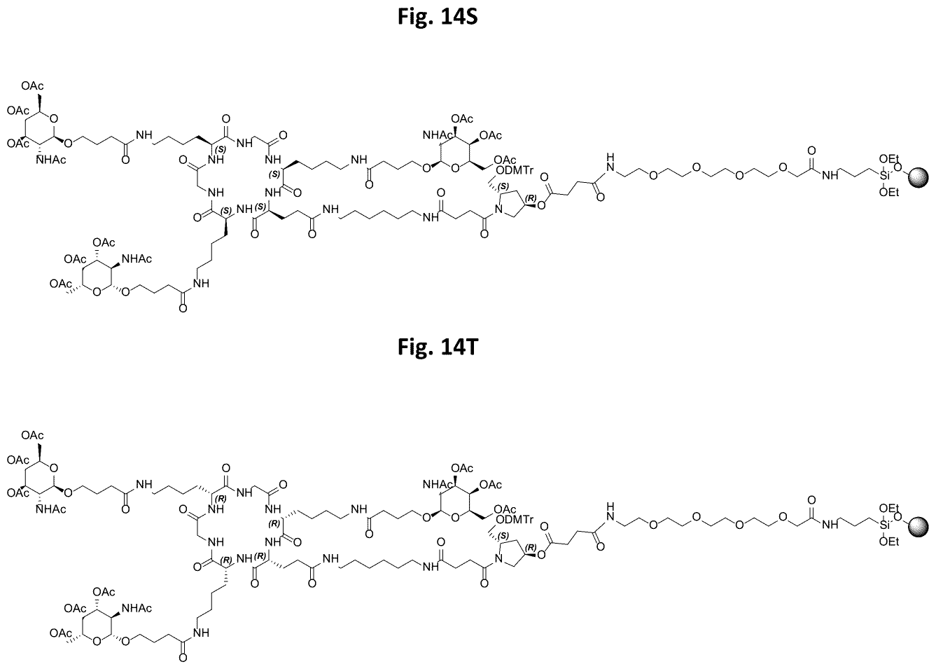

Table 3 provides non-limiting examples of the GalNAc-cyclic peptide conjugates disclosed herein. See also FIGS. 14A-14P for their chemical structures.

TABLE 3
Exemplary GalNAc-Cyclic Peptide Conjugates

| | Peptide | GalNAc | Chemical |
|---|---|---|---|
| Compound ID | scaffold | linker | structure |
| 5-FAM-CPMB-0011 | CPS-001 | Gal-1 | FIG. 14A |
| 5-FAM-CPMB-0012 | CPS-001 | Gal-2 | FIG. 14B |
| 5-FAM-CPMB-0013 | CPS-001 | Gal-3 | FIG. 14C |
| 5-FAM-CPMB-0014 | CPS-001 | Gal-4 | FIG. 14D |
| 5-FAM-CPMB-0015 | CPS-001 | Gal-5 | FIG. 14E |
| 5-FAM-CPMB-0021 | CPS-002 | Gal-1 | FIG. 14F |
| 5-FAM-CPMB-0023 | CPS-002 | Gal-3 | FIG. 14G |
| 5-FAM-CPMB-0025 | CPS-002 | Gal-5 | FIG. 14H |
| 5-FAM-CPMB-0031 | CPS-003 | Gal-1 | FIG. 14I |
| 5-FAM-CPMB-0033 | CPS-003 | Gal-3 | FIG. 14J |
| 5-FAM-CPMB-0034 | CPS-003 | Gal-4 | FIG. 14K |
| 5-FAM-CPMB-0035 | CPS-003 | Gal-5 | FIG. 14L |
| 5-FAM-CPMB-0311 | CPS-031 | Gal-1 | FIG. 14M |
| 5-FAM-CPMB-0313 | CPS-031 | Gal-3 | FIG. 14N |
| CPMB-0013 | CPS-001 | Gal-3 | FIG. 14O |
| CPMB-0023 | CPS-002 | Gal-3 | FIG. 14P |
| 5-FAM-tri-GalNAc | | | FIG. 14Q |
| (positive control) | | | |
| 5-FAM-CPMB-0031- | CPS-003 | Gal-1 | FIG. 14R |
| Ac (negative control) | | | |
| CPMB-0013-DOTMr | CPS-001 | Gal-3 | FIG. 14S |
| CPMB-0023-DOTMr | CPS-002 | Gal-3 | FIG. 14T |

In some examples, the exemplary GalNAc-cyclic peptide conjugate disclosed herein contains the CPS001 cyclic peptide scaffold or the functional equivalent disclosed herein and the GalNAc linker of Gal-3. In other examples, the exemplary GalNAc-cyclic peptide conjugate disclosed herein contains the CPS002 cyclic peptide scaffold or the functional equivalent disclosed herein and the GalNAc linker of Gal-3. In yet other examples, the exemplary GalNAc-cyclic peptide conjugate disclosed herein contains the CPS003 cyclic peptide scaffold or the functional equivalent disclosed herein and the GalNAc linker of Gal-5.

### E. Synthesis of GalNAc-Cyclic Peptide Conjugates

The conjugates described above can be prepared by methods well known in the art, as well as by the synthetic routes disclosed herein. The chemicals used in the synthetic routes may include, for example, solvents, reagents, catalysts, and protecting group and deprotecting group reagents. The methods described herein may also additionally include steps, either before or after the steps described specifically herein, to add or remove suitable protecting groups in order to ultimately allow synthesis of the conjugates or an intermediate thereof. In addition, various synthetic steps may be performed in an alternate sequence or order to give the desired compounds. Synthetic chemistry transformations and protecting group methodologies (protection and deprotection) useful in synthesizing applicable indole compounds are known in the art and include, for example, those described in R. Larock, Comprehensive Organic Transformations, VCH Publishers (1989); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

Briefly, GalNAc moieties coupled with a suitable linker as disclosed herein may be synthesized following routine methods or methods disclosed herein. Separately, cyclic peptide scaffolds as disclosed herein can be prepared following routine methods or methods disclosed herein. The GalNAc moiety and the cyclic peptide scaffold can be reacted at suitable conditions to form a covalent bond, thereby conjugating the GalNAc moieties onto the cyclic peptide scaffold. Similar approaches can be applied for conjugating an agent of interest to the cyclic peptide scaffold via a linker following routine methods or methods disclosed herein.

As a non-limiting example of the synthesis of GalNAc moieties, a suitable lactone moiety can be hydrolyzed to make a terminal hydroxyl-substituted carboxylic acid. The carboxylic acid can be protected and the free hydroxyl substitutes the acetylated hydroxyl on the anomeric carbon of a peracetylated N-glucoseamine. The carboxylic acid can then be deprotected to make the GalNAc coupling partner.

As a non-limiting example of the synthesis of the cyclic peptide, a peptide can be synthesized using known Fmoc-protected solid-phase peptide synthesis with the side chains protected as needed. Peptide synthesis can be followed by derivatization, deprotection of the C- and N-termini, and cyclization of the peptide. Derivatization includes adding an amine linker to a suitable side chain, e.g., glutamic acid. The appropriate amino acid (e.g., lysine) side chains are deprotected and the GalNAc coupling partners are attached using known peptide-bond forming conditions. The amine linker is then used to conjugate agents as desired.

An example of coupling an agent to the amine linker can be 4,4′-dimethoxytrityl (DMTr), which can be used in synthesizing oligonucleotide polymers. By attached a DMTr group to the GalNAc cyclic peptides described above, the resultant conjugate can be used as the substrate for synthesizing an oligonucleotide (e.g., siRNA) using an oligonucleotide synthesizer.

Exemplary synthesis schemes for the cyclic peptide-GalNAc conjugates disclosed herein or any intermediates therein are provided in the Examples below.

### II. Pharmaceutical Compositions

Any of the cyclic peptide-GalNAc conjugates discloses herein, which comprises a diagnostic or therapeutic agent (e.g., a nucleic acid-based agent such as an siRNA molecule) may be formulated into a suitable pharmaceutical composition. The pharmaceutical compositions as described herein can further comprise pharmaceutically acceptable carriers, excipients, or stabilizers in the form of lyophilized formulations or aqueous solutions. Remington: The Science and Practice of Pharmacy 20th Ed. (2000) Lippincott Williams and Wilkins, Ed. K. E. Hoover. Such carriers, excipients or stabilizers may enhance one or more properties of the active ingredients in the compositions described herein, e.g., bioactivity, stability, bioavailability, and other pharmacokinetics and/or bioactivities.

Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; benzoates, sorbate and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, serine, alanine or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ (polysorbate), PLURONICS™ (nonionic surfactants), or polyethylene glycol (PEG).

In some examples, the pharmaceutical composition described herein can be formulated in sustained-release format. Suitable examples of sustained-release preparations include semipermeable matrices of solid hydrophobic polymers, which matrices are in the form of shaped articles, e.g., films, or microcapsules. Examples of sustained-release matrices include polyesters, hydrogels (for example, poly(2-hydroxyethyl-methacrylate), or poly(vinylalcohol), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This can be accomplished by, for example, filtration through sterile filtration membranes. Therapeutic compositions are generally placed into a container having a sterile access port, for example, an intravenous solution bag or vial having a stopper pierceable by a hypodermic injection needle or a sealed container to be manually accessed.

The pharmaceutical compositions described herein can be in unit dosage forms such as solids, solutions or suspensions, or suppositories, for administration by inhalation or insufflation, intrathecal, intrapulmonary or intracerebral routes, oral, parenteral or rectal administration.

For preparing solid compositions, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as powder collections, tablets, pills and capsules. This solid preformulation composition is then subdivided into unit dosage forms of the type described above containing a suitable amount of the active ingredient in the composition.

Suitable surface-active agents include, in particular, non-ionic agents, such as polyoxyethylenesorbitans (e.g., TWEEN 20, 40, 60, 80 or 85) and other sorbitans (e.g., SPAN 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as INTRALIPID™, LIPOSYN™, INFONUTROL™, LIPOFUNDIN™, and LIPIPHYSAN™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. In some embodiments, the compositions are composed of particle sized between 10 nm to 100 mm.

Compositions in preferably sterile pharmaceutically acceptable solvents may be nebulized by use of gases. Nebulized solutions may be breathed directly from the nebulizing device or the nebulizing device may be attached to a face mask, tent, endotracheal tube and/or intermittent positive pressure breathing machine (ventilator). Solution, suspension or powder compositions may be administered, preferably orally or nasally, from devices which deliver the formulation in an appropriate manner.

In some embodiments, any of the pharmaceutical compositions herein may further comprise a second therapeutic agent based on the intended therapeutic uses of the composition.

### III. Delivery of Agents to Liver Cells

Any of the cyclic peptide-GalNAc conjugates disclosed herein can be used for delivering the agent of interest carried by the conjugates (e.g., a diagnostic agent or a therapeutic agent) into liver cells, either in vitro or in vivo. Accordingly, provided herein is a method for delivering an agent of interest into liver cells, the method comprising contacting any of the any of the cyclic peptide-GalNAc conjugates disclosed herein with liver cells to allow for delivery of the agent carried by the conjugate into the liver cells.

In some embodiments, the contacting step can be performed in vitro, e.g., in a cell culture system. For example, an effective amount of the cyclic peptide-GalNAc conjugate as disclosed herein may be incubated with liver cells under suitable culturing conditions for a suitable period of time, allowing for uptake of the conjugate by the liver cells via interaction between the GalNAc moieties and the ASGPR receptor on liver cells. Liver cells containing the conjugate may be enriched and/or expanded. Such liver cells may be administered to a subject for treating a target disease, e.g., those disclosed herein.

Alternatively, any of the cyclic peptide-GalNAc conjugates disclosed herein or a pharmaceutical composition comprising such may be administered to a subject who needs the treatment via suitable route.

To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intratumoral, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a target disease/disorder, for example, a liver disease such as liver cancer. Examples of such target diseases/disorders include acute hepatic porphyria, alagille syndrome, alcohol-related liver disease, alpha-1 antitrypsin deficiency, autoimmune hepatitis, benign liver tumors, biliary atresia, cirrhosis, crigler-najjar syndrome, galactosemia, gilbert syndrome, hemochromatosis, hepatic encephalopathy, hepatitis a, hepatitis b, hepatitis c, hepatorenal syndrome, intrahepatic cholestasis of pregnancy (ICP), lysosomal acid lipase deficiency (LAL-D), liver cysts, liver cancer, newborn jaundice, non-alcoholic fatty liver disease, non-alcoholic steatohepatitis, primary biliary cholangitis (PBC), primary sclerosing cholangitis (PSC), progressive familial intrahepatic cholestasis (PFIC), reye syndrome, type I glycogen storage disease, wilson disease.

A subject having a target disease can be identified by routine medical examination, e.g., laboratory tests, organ functional tests, CT scans, or ultrasounds. In some embodiments, the subject to be treated by the method described herein may be a human cancer patient who has undergone or is subjecting to another therapy, e.g., an anti-cancer therapy, for example, chemotherapy, radiotherapy, immunotherapy, or surgery.

A subject suspected of having any of such target disease/disorder might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Determination of whether an amount of the conjugate achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the conjugate, particularly the agent of interest contained therein, and to prevent the conjugate being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of a conjugate as disclosed herein may be appropriate. Various formulations and devices for achieving sustained release are known in the art.

In one example, dosages for a conjugate as described herein may be determined empirically in individuals who have been given one or more administration(s) of the conjugate. Individuals are given incremental dosages of the agonist. To assess efficacy of the agonist, an indicator of the disease/disorder can be followed.

For the purpose of the present disclosure, the appropriate dosage of a cyclic peptide-GalNAc conjugate as described herein will depend on the specific conjugate, particularly the specific agent of interest carried by the conjugate, the type and severity of the disease/disorder, whether the conjugate is administered for preventive or therapeutic purposes, previous therapy, the patient's clinical history and response to the agonist, and the discretion of the attending physician. Typically the clinician will administer a conjugate, until a dosage is reached that achieves the desired result. Methods of determining whether a dosage resulted in the desired result would be evident to one of skill in the art. Administration of one or more conjugates can be continuous or intermittent, depending, for example, upon the recipient's physiological condition, whether the purpose of the administration is therapeutic or prophylactic, and other factors known to skilled practitioners. The administration of a conjugate disclosed herein may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the conjugate and a physiologically acceptable excipient is infused. Physiologically acceptable excipients may include, for example, 5% dextrose, 0.9% saline, Ringer's solution or other suitable excipients. Intramuscular preparations can be dissolved and administered in a pharmaceutical excipient such as Water-for-Injection, 0.9% saline, or 5% glucose solution.

In one embodiment, a conjugate as disclosed herein can be administered via site-specific or targeted local delivery techniques. Examples of site-specific or targeted local delivery techniques include various implantable depot sources of the conjugate or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Treatment efficacy for a target disease/disorder can be assessed by methods well-known in the art.

### Kits for Use in Treatment of Diseases

The present disclosure also provides kits for use in delivering an agent of interest (e.g., a diagnostic agent or a therapeutic agent) to liver cells, either in vitro or in vivo, and/or for treating or alleviating a target disease. Such kits can include one or more containers comprising a cyclic peptide-GalNAc conjugate, e.g., any of those described herein. In some instances, the conjugate as disclosed herein may be co-used with a second therapeutic agent.

In some embodiments, the kit can comprise instructions for use in accordance with any of the methods described herein. The included instructions can comprise a description of administration of the conjugate, and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein. In still other embodiments, the instructions comprise a description of administering a conjugate as disclosed herein to an individual at risk of the target disease.

The instructions relating to the use of a cyclic peptide-GalNAc conjugate generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as a liver disease, e.g., liver cancer. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a cyclic peptide-GalNAc conjugate as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985>>; Transcription and Translation (B. D. Hames & S. J. Higgins, eds. (1984>>; Animal Cell Culture (R. I. Freshney, ed. (1986>>; Immobilized Cells and Enzymes (IRL Press, (1986>>; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Synthesis of L-Fmoc-Glu(linker-Cbz)-OH and D-Fmoc-Glu(linker-Cbz)-OH

### A: L-Fmoc-Glu(linker-Cbz)-OH

### Step 1: Synthesis of tert-butyl N²-(((9H-fluoren-9-yl)methoxy)carbonyl)-N⁵-(6-(((benzyloxy)carbonyl)amino)hexyl)-L-glutaminate (Compound-1

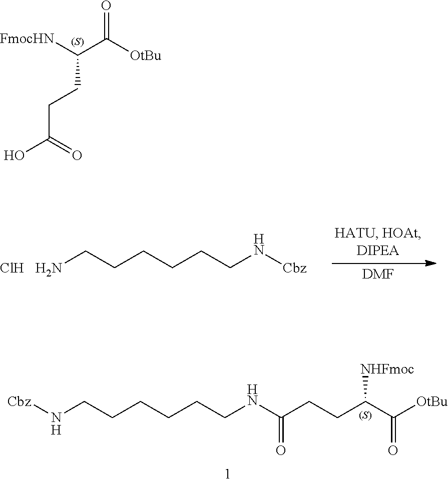

An exemplary synthesis scheme of Compound-1 is provided above. A brief description is provided below.

To a solution of (S)-4-((((9H-fluoren-9-yl)methoxy)carbonyl)amino)-5-(tert-butoxy)-5-oxopentanoic acid (10.0 g, 23.5 mmol, 1.0 equiv.) in DMF (40 mL) was added HATU (8.9 g, 23.5 mmol, 1.0 equiv.), DIPEA (12.3 mL, 70.5 mmol, 3.0 equiv.), HOAt (3.2 g, 23.5 mmol, 1.0 equiv.) in order. The resulting mixture was stirred at 25° C. for 10 min. Once the mixture turned to homogeneous solution, the benzyl (6-aminohexyl)carbamate hydrochloride (8.1 g, 28.2 mmol, 1.2 equiv.) was added and the resulting solution was stirred at 25° C. for 2 h. The reaction progress was monitored by LCMS. Upon completion, the solution was diluted with EtOAc (100 mL) and washed with brine (3×40 mL). The organic layer was dried over Na₂SO₄, filtered and concentrated in vacuo and the crude product was directly used for the next step (yellow solid, 15.5 g).

LCMS: (ESI) m/z=658.2 [M+H]⁺.

### Step 2: N²-(((9H-fluoren-9-yl)methoxy)carbonyl)-N⁵-(6-(((benzyloxy)carbonyl)amino) hexyl)-L-glutamine (L-Fmoc-Glu(linker-Cbz)-OH

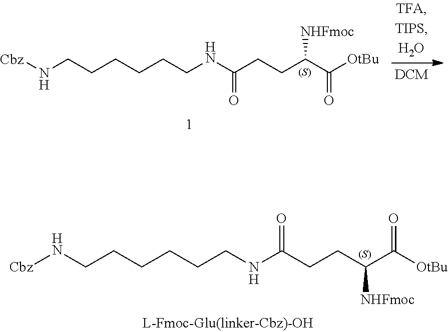

An exemplary synthesis scheme of L-Fmoc-Glu(linker-Cbz)-OH is provided above. A brief description is provided below.

To a solution of compound 1 (15.5 g, 23.5 mmol) in DCM (150 mL) 60 mL of TFA/TIS/H₂O (10/1/1) was added and the resulting solution was stirred overnight at 25° C. and monitored by LCMS. Upon completion, the solution was concentrated in vacuo and the residue was purified by reversed phase chromatography eluting with H₂O (0.01% v/v TFA)/MeCN (95/5 to 5/95). This resulted in 7.0 g (50% yield) of L-Fmoc-Glu(linker-Cbz)-OH as white solid.

LCMS: (ESI) m/z=602.3 [M+H]⁺.

### B: D-Fmoc-Glu(linker-Cbz)-OH

The same procedure described above was used in the synthesis of D-Fmoc-Glu(linker-Cbz)-OH. See also the exemplary synthesis scheme below. From 10.0 g of D-Fmoc-Glu-OtBu, 5.7 g of D-Fmoc-Glu(linker-Cbz)-OH was obtained, total yield was 40%. LCMS: (ESI) m/z=602.1 [M+H]⁺.

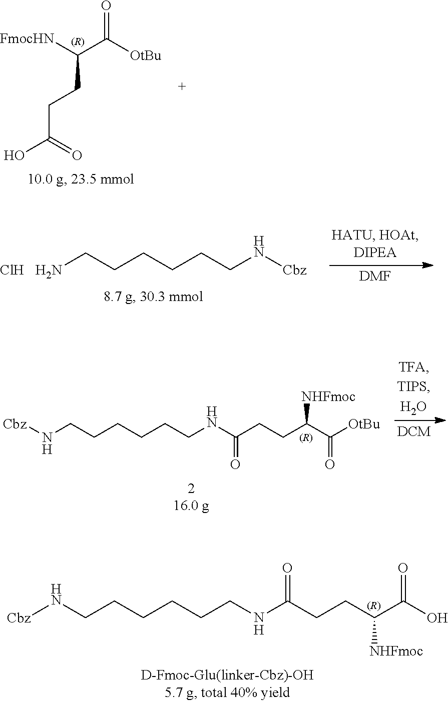

### Example 2: Synthesis of CPMB-001

### Step 1: Synthesis of N²-N²—(N⁵-(6-(((benzyloxy)carbonyl)amino)hexyl)-N²—(N⁶-(tert-butoxycarbonyl)-L-lysyl)-L-glutaminyl)-N⁶-(tert-butoxycarbonyl)-L-lysylglycyl-N⁶-(tert-butoxycarbonyl)-L-lysylglycine (CPMB-001-A

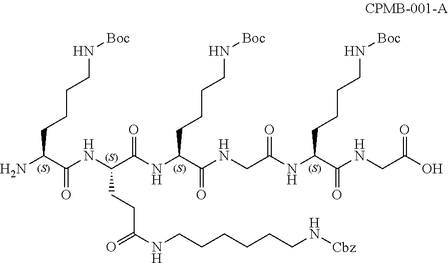

The compound was synthesized by standard solid phase peptide synthesis using the Fmoc strategy.

2-Cl-CTC resin (5.0 g, 1.1 mmol/g, GL Biochem) was swollen in DMF for 10 mins, then the first amino acid Fmoc-Gly-OH (743 mg, 2.5 mmol) was coupled to the resin with DIPEA (1292 mg, 10 mmol) in DMF (80 mL) at room temperature for 4 h. The resin was washed with DCM and de-active excess chloride by MeOH/DCM (1/1, 80 mL) for 1 h. The resin was washed with DMF. The resulting resin was treated with 20% piperidine/DMF (50 mL) for 20 minutes to remove the Fmoc group. The resulting resin was washed with DMF and treated with a solution of Fmoc-Lys(Boc)-OH (1874 mg, 4.0 mmol), HBTU (1517 mg, 4.0 mmol), HOBt (540 mg, 4 mmol) and DIPEA (1034 mg, 8.0 mmol) in DMF (80 mL) at room temperature for 1 h, whereby Lys was introduced to give Fmoc-Lys(Boc)-Gly-CTC resin. In a similar manner with, Gly, Lys(Boc), Glu(tBu), and Lys(Boc) were introduced to give NH₂-Lys(Boc)-Glu(linker-Cbz)-Lys(Boc)-Gly-Lys(Boc)-Gly-CTC resin (SEQ ID NO: 8). The linear peptide was cleaved from resin using cold HFIP/DCM (3/7, 100 mL), to the above dry resin this mixture solution was added and the mixture was shaken for 1.0 h. The resin was filtered off and washed with DCM (10 mL×3). The filtrates were combined and the solvent was removed under vacuo. The crude peptide was dissolved in H₂O/CH₃CN and was lyophilized to remove the remaining solvent. The linear peptide CPMB-001-A was collected as a white solid (1200 mg, crude).

LCMS: (ESI) m/z=540.0 [M+2H]/2+

### Step 2: Synthesis of Benzyl (6-(3-((2S,5S,11S,17S)-5,11,17-tris(4-((tert-butoxycarbonyl)amino)butyl)-3,6,9,12,15,18-hexaoxo-1,4,7,10,13,16-hexaazacyclooctadecan-2-yl)propanamido)hexyl)carbamate (CPMB-001-B

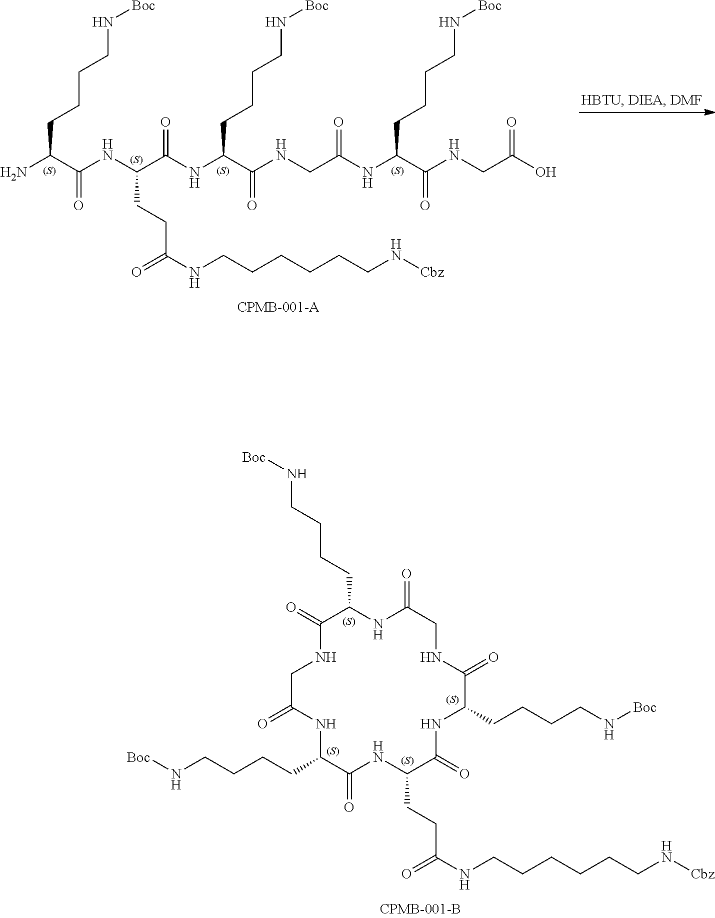

An exemplary synthesis scheme for producing CPMB-001-B from CPMB-001-A is provided above. Below is a brief description of the synthesis procedures.

HBTU (774 mg, 2.04 mmol, 2.0 eq.) was dissolved in DMF (20 mL) and DIEA (168 μL) was added. A solution of CPMB-001-A (1200 mg, 1.02 mmol, 1.0 eq.) and DIEA (506 μL) in DMF (150 mL) was then added dropwise to the HBTU solution over a period of 2 h at room temperature. A brown mixture was obtained, and LCMS indicated entire conversion of CPMB-001-A. The reaction was then quenched by addition of water (170 mL). The resulting mixture was extracted with ethyl acetate (200 mL×3). The organic layer was washed with a solution of NaCl (brine 100 mL with water 100 mL), dried over anhydrous Na₂SO₄ and concentrated under reduced pressure to obtain pale-brown oil. The oil was triturated in water (100 mL) and filtered. The residue was triturated again in n-hexane (100 mL, containing 5% ethyl acetate) for 2 h and filtered to obtain the target cyclic peptide as pale-yellow solid (742 mg, yield: 62.7%).

LCMS: (ESI) m/z=1162.9 [M+H]⁺.

### Step 3: Synthesis of Benzyl (6-(3-((2S,5S,11S,17S)-5,11,17-tris(4-aminobutyl)-3,6,9,12,15,18-hexaoxo-1,4,7,10,13,16-hexaazacyclooctadecan-2-yl)propanamido)hexyl)carbamate (CPMB-001

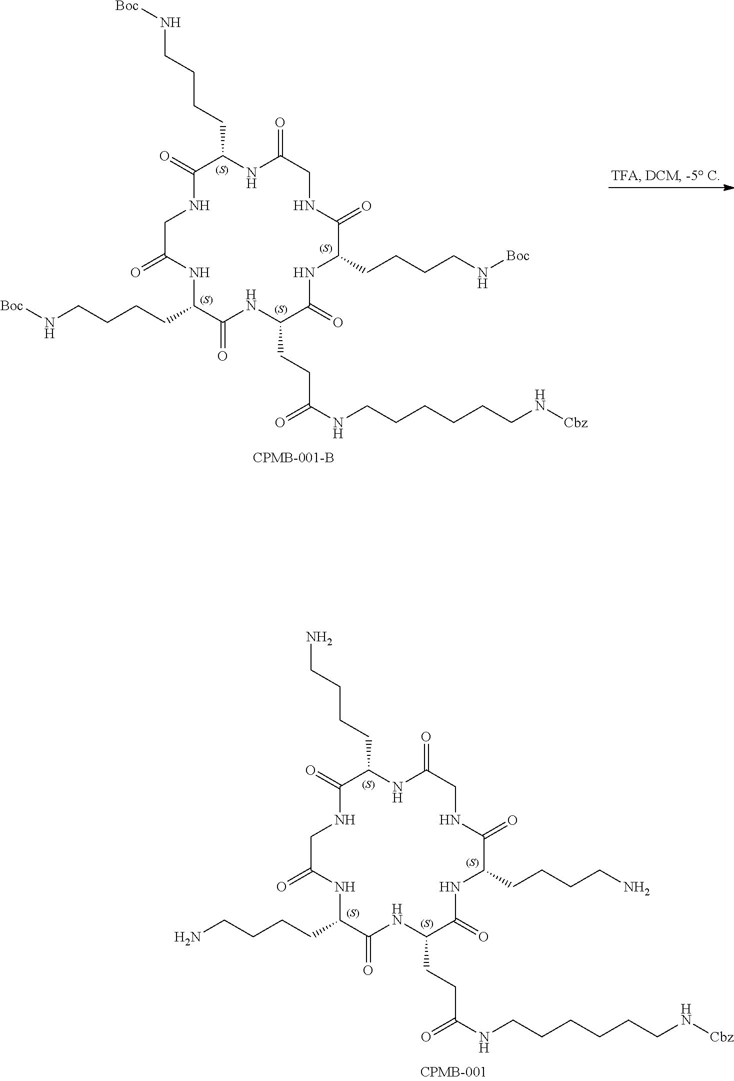

An exemplary synthesis scheme for producing CPMB-001 from CPMB-001-B is provided above. Below is a brief description of the synthesis procedures.

The CPMB-001-B (742 mg, 0.64 mmol) was dissolved in DCM (1 mL) and cooled to −5° C. and then iced TFA/DCM (2 mL, v:v=1:1) was added. The solution was stirred at −5° C. for 1 h and the reaction was monitored by LCMS. After the complete conversion of CPMB-001-B, iced MTBE (40 mL) was added and white sediment was generated. The suspension was centrifuged at 3200 r/min for 3 min and the supernatant was poured out. The sediment was washed with MTBE (40 mL×2) and centrifuged for two extra times. The white residue was dried under reduced pressure to obtain the target compound (CPMB-001) as white solid (760 mg salt, yield: 98.8%).

LCMS: (ESI) m/z=860.3 [M+H]⁺.

### Example 3: Synthesis of CPMB-002 and CPMB-003

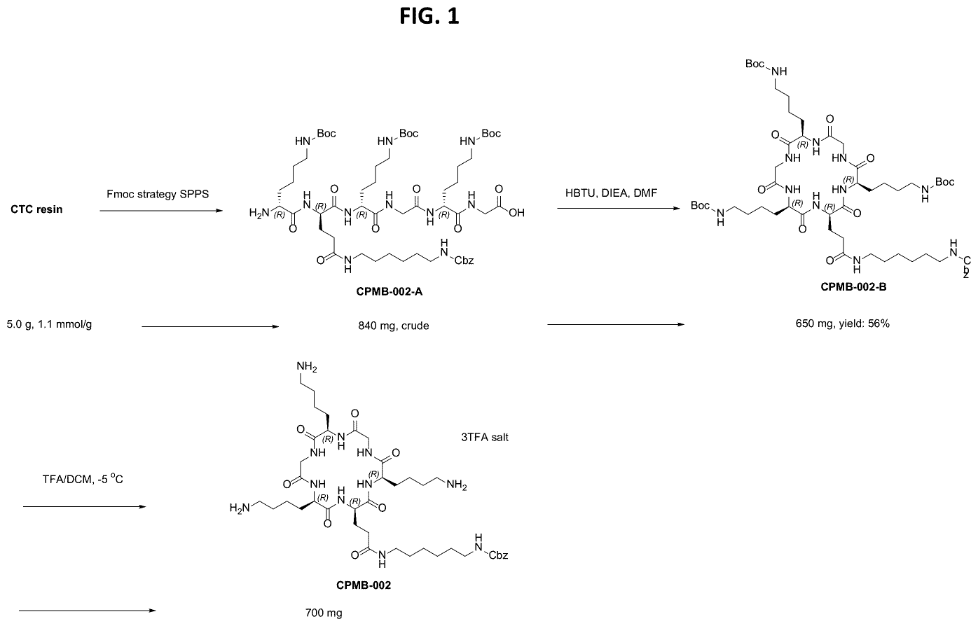

The same procedure was used in the synthesis of CPMB-002. See the exemplary synthesis scheme in FIG. 1.

From 5.0 g (1.1 mmol) of CTC resin, 700 mg of was CPMB-002 obtained, total yield was 50%. LCMS: (ESI) m/z=860.3 [M+H]⁺.

From 5.0 g (1.1 mmol) of CTC resin, 781 mg of CPMB-003 was obtained, total yield was 65.0%. LCMS: (ESI) m/z=860.3 [M+H]⁺.

### Example 4: Synthesis of Gal-1

### Gal-1: 5-(((2R,3R,4R,5R,6R)-3-acetamido-4,5-diacetoxy-6-(acetoxymethyl) tetrahydro-2H-pyran-2-yl)oxy)pentanoic acid (Gal-1

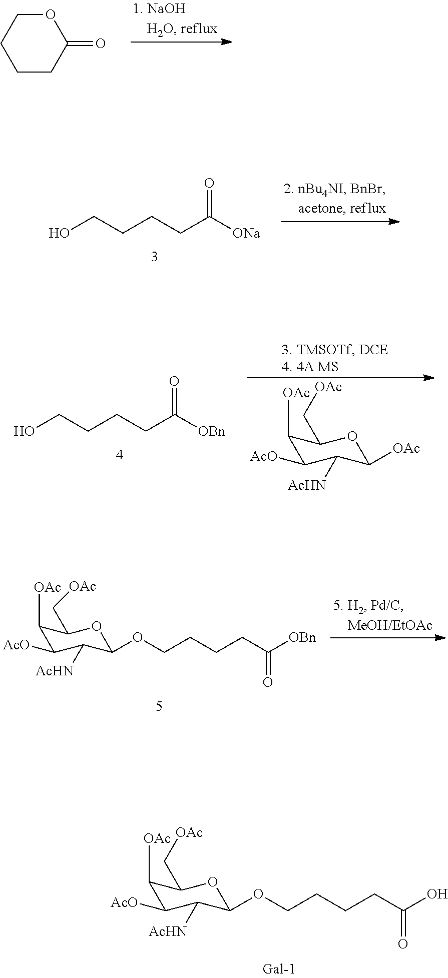

An exemplar synthesis scheme for Gal-1 is provided above.

### Step 1 and 2: Synthesis of sodium 5-hydroxypentanoate (Compound 3) and benzyl 5-hydroxypentanoate (Compound 4)

The mixture of tetrahydro-2H-pyran-2-one (10.0 g, 99.9 mmol, 1.0 equiv.) and NaOH (4.0 g, 99.9 mmol, 1.0 equiv.) were dissolved in H₂O (100 mL). The solution was refluxed at 100° C. overnight and monitored by LCMS. Upon completion, the solution was concentrated in vacuo. The resulting white solid was taken up in acetone (200 mL) and nBu₄NI (1.8 g, 5.0 mmol, 5 mol %) and benzyl bromide (14.2 mL, 119.9 mmol, 1.2 equiv.) were added in order. The mixture was refluxed at 60° C. overnight and monitored by LCMS. Upon completion, the solution was concentrated in vacuo. The subsequent residue was dissolved in EtOAc (150 mL) and washed with aqueous NaHSO₄ (10.0 g in 150 mL H₂O). The aqueous layer was then extracted with EtOAc (3×50 mL). The combined organic layer was washed with saturated aqueous NaHCO₃ (100 mL), brine (40 mL), dried over Na₂SO₄, filtered and concentrated in vacuo. The residue was purified by silica gel column eluting with PE/EtOAc (8/2). This resulted in 17.9 g (86% yield) of benzyl 5-hydroxypentanoate (Compound 4) as colorless oil. LCMS: (ESI) m/z=209.1 [M+H]⁺.

### Step 3: Synthesis of (2R,3R,4R,5R,6R)-5-acetamido-2-(acetoxymethyl)-6-((5-(benzyloxy)-5-oxopentyl)oxy)tetrahydro-2H-pyran-3,4-diyl diacetate (Compound 5)

To a suspension of (2S,3R,4R,5R,6R)-3-acetamido-6-(acetoxymethyl)tetrahydro-2H-pyran-2,4,5-triyl triacetate (10.0 g, 25.7 mmol, 1.0 equiv.) in dry DCE (200 mL) was added trimethylsilyl trifluoromethanesulfonate (7.0 mL, 38.5 mmol, 1.5 equiv.). The mixture was stirred for 2 h at 25° C., followed by Compound 4 (7.5 g, 36.0 mmol, 1.4 equiv.) and 4 Å molecular sieve (5.0 g). The mixture was stirred overnight at 25° C. and monitored by LCMS. Once the reaction was completed, the mixture was filtered to remove the 4 Å molecular sieve. The filtrate was added aqueous saturated NaHCO₃ (50 mL) and extracted with DCM (3×50 mL). The combined organic layer was washed with brine (40 mL), dried over Na₂SO₄, filtered and concentrated in vacuo. The subsequent yellow oil was directly used for the next step. LCMS: (ESI) m/z=538.0 [M+H]⁺.

### Step 4: Synthesis of 5-(((2R,3R,4R,5R,6R)-3-acetamido-4,5-diacetoxy-6-(acetoxymethyl) tetrahydro-2H-pyran-2-yl)oxy)pentanoic acid (Gal-1

To the solution of Compound 5 in MeOH/EtOAc (10.0 mL/30.0 mL) was added Pd/C (1.0 g, 10.0%). The flask was evacuated and flushed 3 times with H₂. The suspension was stirred overnight at 25° C. and monitored by LCMS. Upon completion, the solution was filtered and concentrated in vacuo. The residue was purified by reversed phase chromatography eluting with H₂O (0.01% v/v TFA)/MeCN (95/5 to 5/95). This resulted in 7.4 g (64% yield) of Gal-1 as white foam.

LCMS: (ESI) m/z=448.0 [M+H]⁺.

¹H NMR (400 MHz, DMSO) δ 12.02 (br, 1H), 7.82 (d, J=9.2 Hz, 1H), 5.21 (d, J=3.4 Hz, 1H), 4.96 (dd, J=11.2, 3.4 Hz, 1H), 4.48 (d, J=8.5 Hz, 1H), 4.03 (s, 3H), 3.88 (dt, J=11.2, 8.9 Hz, 1H), 3.73-3.66 (m, 1H), 3.46-3.37 (m, 1H), 2.20 (t, J=7.1 Hz, 2H), 2.11 (s, 3H), 2.00 (s, 3H), 1.89 (s, 3H), 1.77 (s, 3H), 1.54-1.43 (m, 4H).

### Example 5: Synthesis of Gal-2

### Gal-2: Synthesis of 3-(2-(((2R,3R,4R,5R,6R)-3-acetamido-4,5-diacetoxy-6-(acetoxymethyl)tetrahydro-2H-pyran-2-yl)oxy)ethoxy)propanoic acid (Gal-2

The same procedure is used in the synthesis of Gal-1. An exemplary synthesis scheme is provided below. From 5.1 g (43.9 mmol) of 1,4-dioxepan-5-one, 0.8 g of Gal-2 was obtained, the total yield was 4.1%.

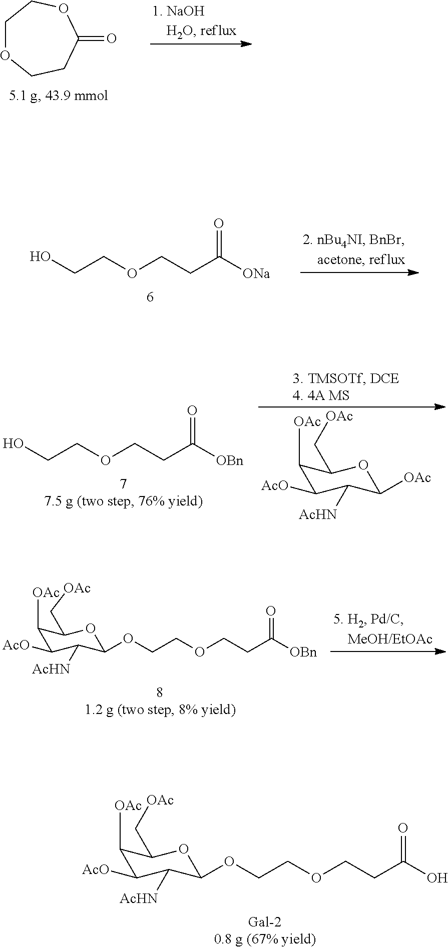

LCMS: (ESI) m/z=464.4 [M+H]⁺.

### Example 6: Synthesis of Gal-3

### Gal-3: Synthesis of 4-(((2R,3R,4R,5R,6R)-3-acetamido-4,5-diacetoxy-6-(acetoxymethyl)tetrahydro-2H-pyran-2-yl)oxy)butanoic acid (Gal-3

The same procedure is used in the synthesis of Gal-1. A synthesis scheme is provided below. From 15.0 g (174.2 mmol) of dihydrofuran-2(3H)-one, 3.9 g of Gal-3 as white foam was obtained, the total yield was 5.2%.

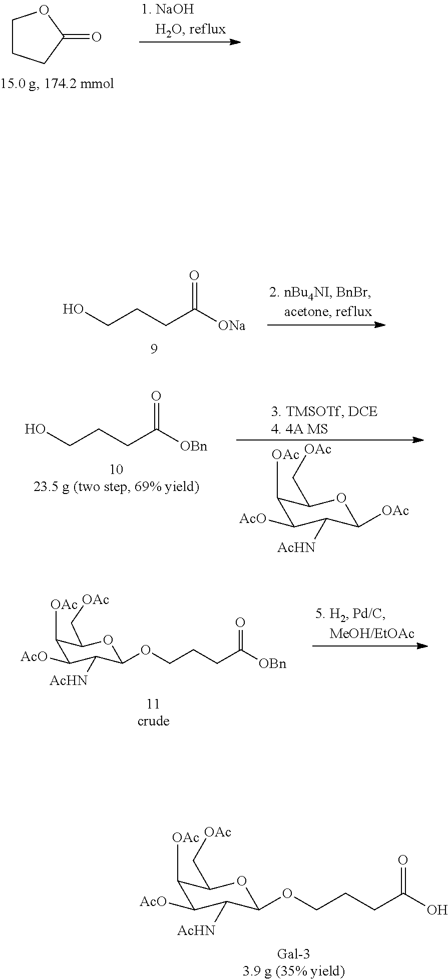

LCMS: (ESI) m/z=434.2 [M+H]⁺.

¹H NMR (400 MHz, DMSO) δ 12.03 (br, 1H), 7.83 (d, J=9.2 Hz, 1H), 5.75 (s, 1H), 5.21 (d, J=3.4 Hz, 1H), 4.95 (dd, J=11.3, 3.4 Hz, 1H), 4.47 (d, J=8.5 Hz, 1H), 4.07-3.98 (m, 3H), 3.87 (dt, J=11.3, 8.9 Hz, 1H), 3.70 (dt, J=10.0, 6.1 Hz, 1H), 2.23 (t, J=7.4 Hz, 2H), 2.10 (s, 3H), 1.99 (s, 3H), 1.89 (s, 3H), 1.77 (s, 3H), 1.72-1.64 (m, 2H).

### Example 7: Synthesis of Gal-4

### Gal-4: Synthesis of 6-(((2R,3R,4R,5R,6R)-3-acetamido-4,5-diacetoxy-6-(acetoxymethyl) tetrahydro-2H-pyran-2-yl)oxy)hexanoic acid (Gal-4

The same procedure is used in the synthesis of Gal-1. An exemplary synthesis scheme is provided below. From 11.4 g (99.9 mmol) of oxepan-2-one, 10.6 g of Gal-4 as white foam was obtained, the total yield was 23%.

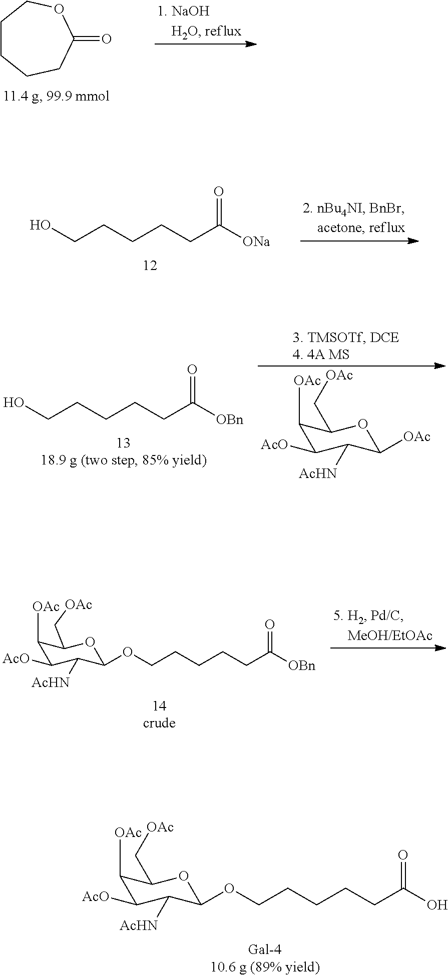

LCMS: (ESI) m/z=462.2 [M+H]⁺.

¹H NMR (400 MHz, DMSO) δ 12.07 (br, 1H), 7.82 (d, J=9.2 Hz, 1H), 5.21 (d, J=3.4 Hz, 1H), 4.96 (dd, J=11.2, 3.4 Hz, 1H), 4.48 (d, J=8.5 Hz, 1H), 4.05-4.01 (m, 3H), 3.87 (dt, J=11.2, 8.9 Hz, 1H), 3.69 (dt, J=9.9, 6.4 Hz, 1H), 3.41 (dt, J=9.9, 6.4 Hz, 1H), 2.18 (t, J=7.4 Hz, 2H), 2.11 (s, 3H), 2.00 (s, 3H), 1.89 (s, 3H), 1.77 (s, 3H), 1.54-1.41 (m, 4H), 1.30-1.24 (m, 2H).

### Example 8: Synthesis of Gal-5

### Gal-5: Synthesis of 3-(((2R,3R,4R,5R,6R)-3-acetamido-4,5-diacetoxy-6-(acetoxymethyl)tetrahydro-2H-pyran-2-yl)oxy)propanoic acid (Gal-5

The same procedure is used in the synthesis of Gal-1. An exemplary synthesis scheme is provided below From 6.5 g (36 mmol) of benzyl 3-hydroxypropanoate, 4.3 g of Gal-5 as white foam was obtained, the total yield was 28.5%.

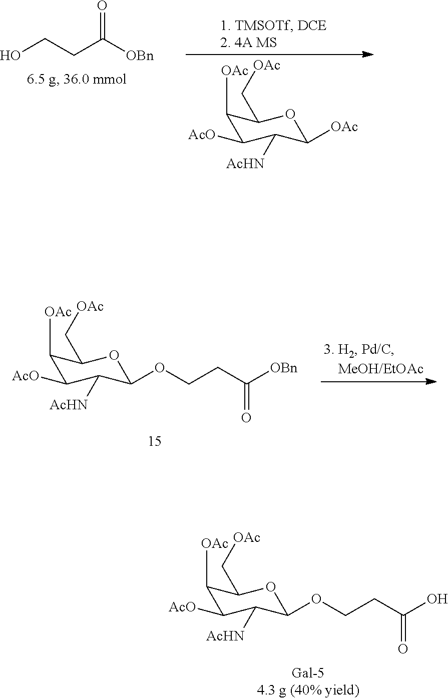 [SMILES: O=C(CCO)OCc1ccccc1 ; CC(=O)NC1C(OC(C)=O)OC(COC(C)=O)C(OC(C)=O)C1OC(C)=O ; CC(=O)NC1C(OCCC(=O)OCc2ccccc2)OC(COC(C)=O)C(OC(C)=O)C1OC(C)=O ; CC(=O)NC1C(OCCC(=O)O)OC(COC(C)=O)C(OC(C)=O)C1OC(C)=O]

LCMS: (ESI) m/z=420.0 [M+H]⁺.

¹H NMR (400 MHz, DMSO) δ 12.27 (br, 1H), 7.77 (d, J=9.2 Hz, 1H), 5.21 (d, J=3.4 Hz, 1H), 4.96 (dd, J=11.2, 3.4 Hz, 1H), 4.53 (d, J=8.5 Hz, 1H), 4.08-3.98 (m, 3H), 3.92-3.80 (m, 2H), 3.68 (dt, J=10.3, 6.4 Hz, 1H), 2.46 (t, J=6.4 Hz, 2H), 2.10 (s, 3H), 2.00 (s, 3H), 1.89 (s, 3H), 1.77 (s, 3H).

### Example 9: Synthesis of 5-Fam-CPMB-0013

### Step 1: Synthesis of CPMB-0013-A

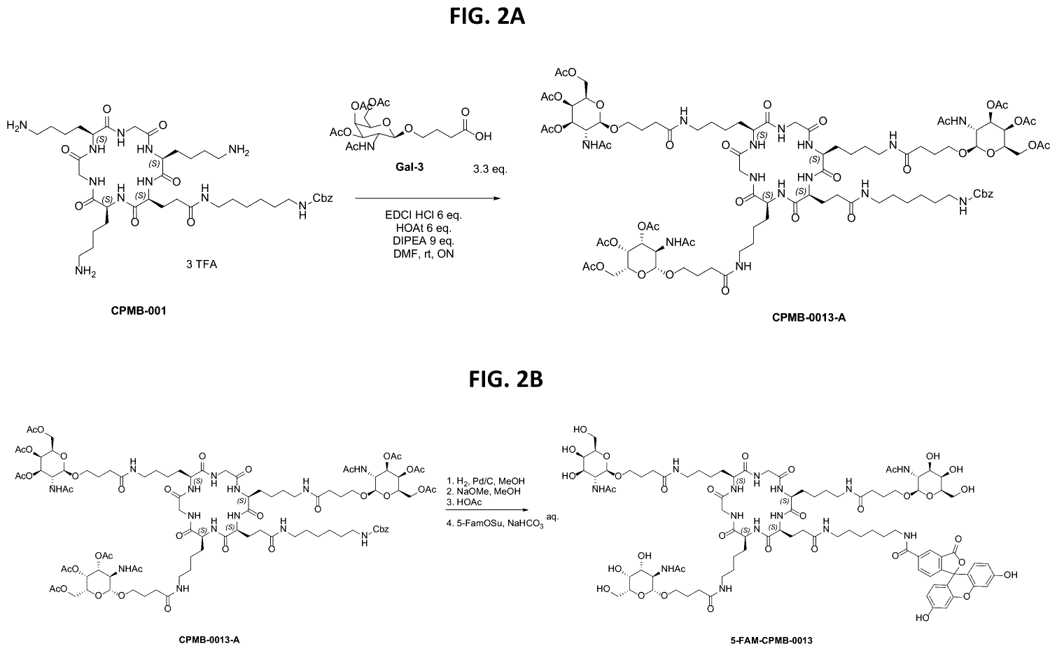

An exemplary synthesis scheme for producing CPMB-0013A is provided in FIG. 2A.

To a solution of benzyl (6-(3-((2S,5S,11S,17S)-5,11,17-tris(4-aminobutyl)-3,6,9,12,15,18-hexaoxo-1,4,7,10,13,16-hexaazacyclooctadecan-2-yl)propanamido)hexyl)carbamate (CPMB-001) (898 mg, 747 μmol, 1.0 equiv.) in DMF (5 mL) was added EDCI·HCl (859 mg, 4.48 mmol, 6.0 equiv.), HOAt (610 mg, 4.48 mmol, 6.0 equiv.), DIPEA (1.17 mL, 6.72 mmol, 9.0 equiv.), 4-(((2R,3R,4R,5R,6R)-3-acetamido-4,5-diacetoxy-6-(acetoxymethyl)tetrahydro-2H-pyran-2-yl)oxy) butanoic acid (Gal-3) (1.07 g, 2.47 mmol, 3.3 equiv.) in order. The resulting solution was stirred at 25° C. overnight and monitored by LCMS. Upon completion, the solution was diluted with H₂O (15 mL), extracted with DCM (3×20 mL), dried over Na₂SO₄, filtered and concentrated in vacuo. The residue was purified by reversed phase chromatography eluting with H₂O (0.01% v/v TFA)/MeCN (95/5 to 5/95) to afford the compound CPMB-0013-A as white foam (1.22 g, 78% yield).

### Purification Method:

Mobile Phase: A: 0.05% TFA in water; B: 0.05% TFA in acetonitrile
Column: Waters XBridge Prep C18, 19×250 mm, 10 μm, 110 Å
Flow rate: 25 mL/min
Eluants: elution on a lineal density gradient of A/B=71/29-61/39 (20 mins)

LCMS: (ESI) m/z=1053.3 [M/2+H]⁺.

### Step 2: Synthesis of 5-Fam-CPMB-0013

An exemplary synthesis scheme for producing 5-Fam-CPMB-0013 is provided in FIG. 2B.

To a solution of CPMB-0013-A (14.3 mg, 6.8 μmol, 1.0 equiv.) in MeOH (2.0 mL) was added Pd/C (4.0 mg, 10.0%). The flask was evacuated and flushed 3 times with H₂. The resulting mixture was stirred for 16 h at 25° C. and monitored by LCMS. Upon completion, the mixture was filtered through a syringe equipped with a filter membrane (0.5 μm, NAVIGATOR©). To the filtrate was added a solution of NaOMe in MeOH (100 μL, 30.0 wt %, 5.4 M). The resulting solution was stirred for 20 min at 25° C. and monitored by LCMS. Upon completion, the solution was neutralized by addition of acetic acid (31.0 μL). The solution was concentrated in vacuo and the residue was dissolved in saturated aqueous NaHCO₃ (1 mL) was diluted with H₂O (1.0 mL), then added 2,5-dioxopyrrolidin-1-yl 3′,6′-dihydroxy-3-oxo-3H-spiro[isobenzofuran-1,9′-xanthene]-5-carboxylate (5FAM-OSu, 4.8 mg, 10.2 μmol, 1.5 equiv.). The flask was covered with the aluminum foil to prevent the solution from light. The resulting solution was stirred for 16 h at 25° C. and monitored by LCMS. Upon completion, the solution was diluted with MeCN (2.0 mL) and purified by prep-HPLC to afford 5-Fam-CPMB-0013 as orange solid (5.0 mg, 38% yield).

### Purification Method:

Mobile Phase: A: 0.05% TFA in water; B: 0.05% TFA in acetonitrile
Column: Phenomenex Gemini C18, 21.2×250 mm, 10 m, 110 Å
Flow rate: 25 mL/min
Eluants: elution on a lineal density gradient of A/B=84/16-74/26 (20 mins)

LCMS: (ESI) m/z=976.2 [M+2H]/2⁺.

HPLC: 97.57% (214 nm), RT=12.776 min

Mobile Phase: A: Water (0.05% TFA) B: ACN (0.05% TFA)

Gradient: 5% B for 3 min, increase to 65% B within 20 min, increase to 95% within 2 min, hold for 5 min, back to 5% B within 0.1 min.

Flow Rate: 1.0 mL/min

Column: XBridge peptide BEH column C18, 4.6×150 mm, 3.5 μm, 130 Å

Column Temperature: 20° C.

### Example 10: Synthesis of CPMB-0013

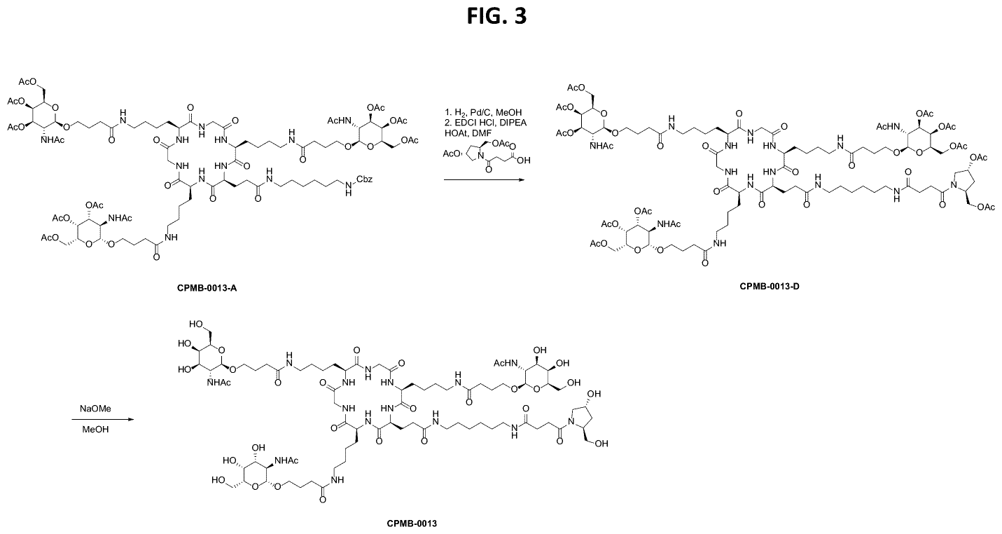

An exemplary synthesis scheme for producing CPMB-0013 is provided in FIG. 3.

To a solution of CPMB-0013-A (695 mg, 0.33 mmol, 1.0 equiv.) in MeOH (20.0 mL) was added Pd/C (0.18 g, 10.0%). The flask was evacuated and flushed 3 times with H₂. The resulting mixture was stirred for 16 h at 25° C. and monitored by LCMS. Upon completion, the mixture was filtered and concentrated. The residue was dissolved in 10 mL DMF and EDCI·HCl (94.8 mg, 0.49 mmol, 1.5 equiv.), HOAt (67.3 mg, 0.49 mmol, 1.5 equiv.), DIPEA (173 μL, 1 mmol, 3.0 equiv.), and 4-((2S,4R)-4-acetoxy-2-(acetoxymethyl)pyrrolidin-1-yl)-4-oxobutanoic acid (4-((2S,4R)-4-acetoxy-2-(acetoxymethyl) pyrrolidin-1-yl)-4-oxobutanoic acid: 119.2 mg, 0.39 mmol, 1.2 equiv.) were added in order. The reaction was stirred at rt for 3 h and monitored by LCMS. After the completion of reaction, the mixture was directly purified by reversed phase chromatography eluting with H₂O (0.01% v/v TFA)/MeCN (95/5 to 5/95) to afford the compound CPMB-0013-D as white powder. (0.65 g, 87% yield).

The compound CPMB-0013-D (0.65 g, 0.29 mmol, 1.0 eq.) was dissolved in 20 mLMeOH and a solution of NaOMe in MeOH (30 wt %, 500 μL) was added. The solution was stirred at rt for 20 min and the LCMS indicated that the deacetylation process was completed. The acetic acid (155 μL) was then added to the mixture to neutralize the solution. The mixture was diluted with water (15 mL) and purified with prep-HPLC eluting with H₂O (10 mmol NH₄OAc)/MeCN to afford 280 mg desired compound CPMB-0013 as white foam. (54% yield)

### Purification Method:

Mobile Phase: A: 10 mmol NH₄OAc; B: ACN
Column: Waters XBridge Prep C18, 19×250 mm, 10 μm, 130 Å
Flow rate: 25 mL/min
Eluants: elution on a lineal density gradient of A/B=95/5-85/15 (20 mins), fraction at 20. 92 min was collected and lyophilized
LCMS: (ESI) m/z=795.7 [(M-GalNAc)/2+H]+
HPLC: 90.04% (214 nm), RT=12.28 min
   Mobile Phase: A: Water (0.01% TFA) B: ACN (0.01% TFA)
   Gradient: 2% B for 4 min, increase to 32% B within 15 min, increase to 95% B within 3 min, hold for 5 min, back to 5% B within 0.1 min.
   Flow Rate: 1 mL/min
   Column: XBridge Peptide BEH C18, 4.6×150 mm, 3.5 μm, 130 Å
   Column Temperature: 40° C.

### Example 11: Synthesis of CPMB-0013-DMTr

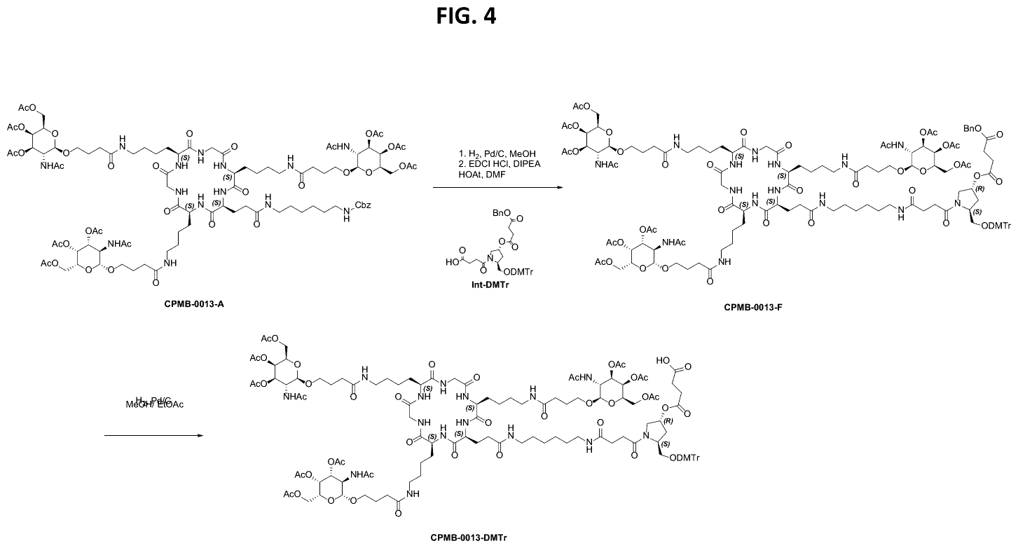

An exemplary synthesis scheme for producing CPMB-0013-DMTr is provided in FIG. 4.

### Step 1: Synthesis of CPMB-0013-F

To a solution of CPMB-0013-A (210 mg, 0.10 mmol, 1.0 equiv.) in MeOH (20.0 mL) was added Pd/C (50 mg, 10.0%). The flask was evacuated and flushed 3 times with H₂. The resulting mixture was stirred for 16 h at 25° C. and monitored by LCMS. Upon completion, the mixture was filtered through a syringe equipped with a filter membrane (0.5 μm, NAVIGATOR©). The residue was dissolved in 1 mL DMF and EDCI·HCl (28.6 mg, 0.15 mmol, 1.5 equiv.), HOAt (20.3 mg, 0.15 mmol, 1.5 equiv.), DIPEA (52 μL, 0.30 mmol, 3.0 equiv.), and Int-DMTr (84.6 mg, 0.12 mmol, 1.2 equiv.) were added in order. The reaction was stirred at rt for 3 h and monitored by LCMS. After the completion of reaction, the mixture was directly purified by reversed phase chromatography eluting with H₂O (0.01% v/v NH₄HCO₃)/MeCN (95/5 to 5/95) to afford the compound CPMB-0013-F as white powder. (177 mg, 67% yield).

### Purification Method:

Mobile Phase: A: 10 mmol NH₄OAc; B: ACN
Column: Waters XBridge Prep C18, 19×250 mm, 10 μm, 130 Å
Flow rate: 25 mL/min
Eluants: elution on a lineal density gradient of A/B=56/44-46/54 (20 mins), fraction at 19.52 min with 100% was collected and lyophilized
LCMS: (ESI) m/z=1181.4 [(M-DMTr)/2+H]+

### Step 2: Synthesis of CPMB-0013-DMTr

To the solution of CPMB-0013-F in MeOH/EtOAc (20.0 mL, 1:1) was added Pd/C (40 mg, 10.0%). The flask was evacuated and flushed 3 times with H₂. The suspension was stirred for 6 h at 25° C. and monitored by LCMS. Upon completion, the solution was filtered and concentrated in vacuo. The residue was purified by prep-HPLC eluting with H₂O (0.01% v/v NH₄HCO₃)/MeCN (95/5 to 5/95) to afford 33 mg desired compound CPMB-0013-DMTr as white powder. (19% yield)

### Purification Method:

Mobile Phase: A: 10 mmol NH₄OAc; B: ACN
Column: Waters XBridge Prep C18, 19×250 mm, 10 μm, 130 Å
Flow rate: 25 mL/min
Eluants: elution on a lineal density gradient of A/B=68/32-58/42 (20 mins), fraction at 15.65 min with 98.4% was collected and lyophilized

LCMS: (ESI) m/z=1285.6 [M/2−H]⁻.

HPLC: >99% (214 nm), RT=10.58 min
Mobile Phase: A: Water (10 mM NH₄HCO₃) B: ACN
Gradient: 5% B for 1 min, increase to 95% B within 20 min, increase to 95% B for 5 min, back to 5% B within 0.1 min.
Flow Rate: 1 mL/min
Column: XBridge Peptide BEH C18, 4.6×150 mm, 3.5 μm, 130 Å
Column Temperature: 40° C.

### Example 12: Synthesis of CPMB-0013-DTMr-PEG4-CPG Resin

### Step 1: Synthesis of CPG-PEG4

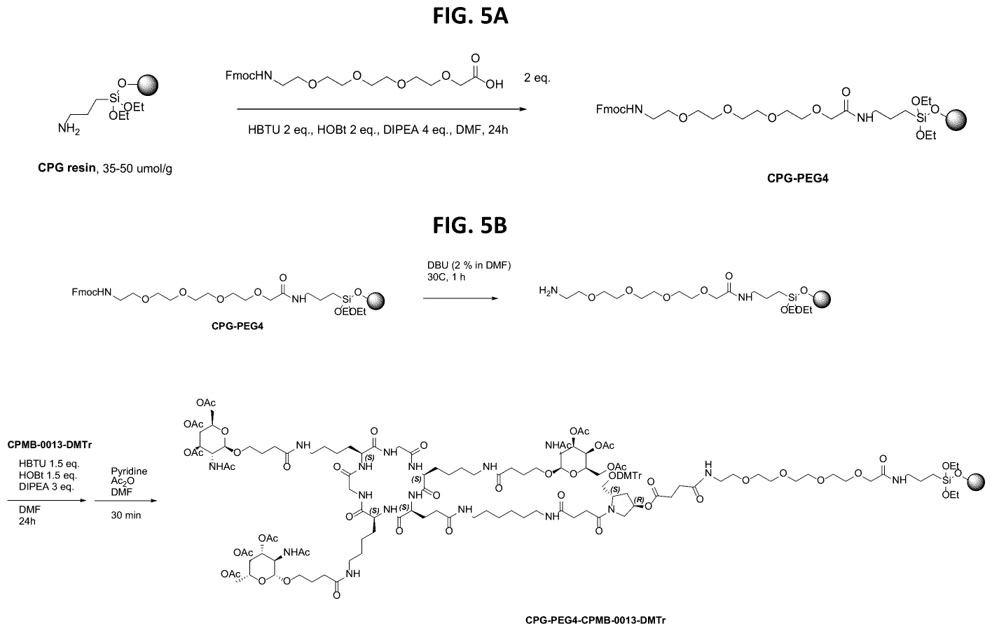

An exemplary synthesis scheme for producing CPG-PEG4 is provided in FIG. 5A.

Shake the CPG resin (488 mg, 35-50 μmol/g), 1-(9H-fluoren-9-yl)-3-oxo-2,7,10,13,16-pentaoxa-4-azaoctadecan-18-oic acid (FmocNH-PEG4-CH₂COOH) (23 mg, 48.8 μmol, 2.0 equiv.), DIPEA (17 μL, 97.6 μmol, 4.0 equiv.), HOBt (6.6 mg, 48.8 μmol, 2.0 equiv.) and HBTU (18.5 mg, 48.8 μmol, 2.0 equiv.) in DMF (2 mL) at room temperature for 24 h. Then wash the resin with DMF (3 mL×3). Kaiser test negative. The resin was then washed with MTBE (5 mL×3) and dried in vacuo. Loading test: take 6.1 mg, 8.0 mg, 12.0 mg CPG resin (m_{CPG}) respectively in 3 EP tubes and add 1 mL DBU (2% in DMF) in each tube, after 30 min, transfer 800 μL of supernatant in 25 mL volumetric flask and fill the flask with MeCN. The spectrophotometry allowed determining the absorbance of Fmoc group (Avg_{304 nm}): 0.069, 0.091, 0.138. Thus, the loading is Avg*4.1/m_{CPG}=47 μmol/g, >99% yield.

### Step 2: Synthesis of CPG-PEG4-CPMB-0013-DMTr

An exemplary synthesis scheme for producing CPG-PEG4-CPMB-0013-DMTr is provided in FIG. 5B.

Shake the CPG-PEG4 resin (150 mg, 47 μmol/g), in 3 mL DBU (2% in DMF) for 1 h. Kaiser test indicated the completion of de-Fmoc process, then wash the resin with DMF (3 mL×3). Shake the resin with CPMB-0013-DMTr (27 mg, 10.5 μmol, 1.5 equiv.), DIPEA (3.6 μL, 10.5 μmol, 3.0 equiv.), HOBt (1.4 mg, 10.5 μmol, 1.5 equiv.) and HBTU (4 mg, 10.5 μmol, 1.5 equiv.) in DMF (2 mL) at room temperature for 24 h. Then wash the resin with DMF (3 mL×3). Charge the resin with pyridine/Ac₂O (1 mL/20 μL) and shake for 1 h. The resin was then washed with DMF (3 mL×3) and MTBE (5 mL×3) and dried in vacuo. Loading test: take 2.7 mg CPG-PEG4-CPMB-0013-DMTr resin, add 200 μL 1N HCl and 200 μL MeCN, LCMS: S(DMTr⁺)=691.54 mAU, loading=32 μmol/g, 51.6 mg.

### Example 13: Synthesis of 5-Fam-CPMB-0023

### Step 1: Synthesis of CPMB-0023-A

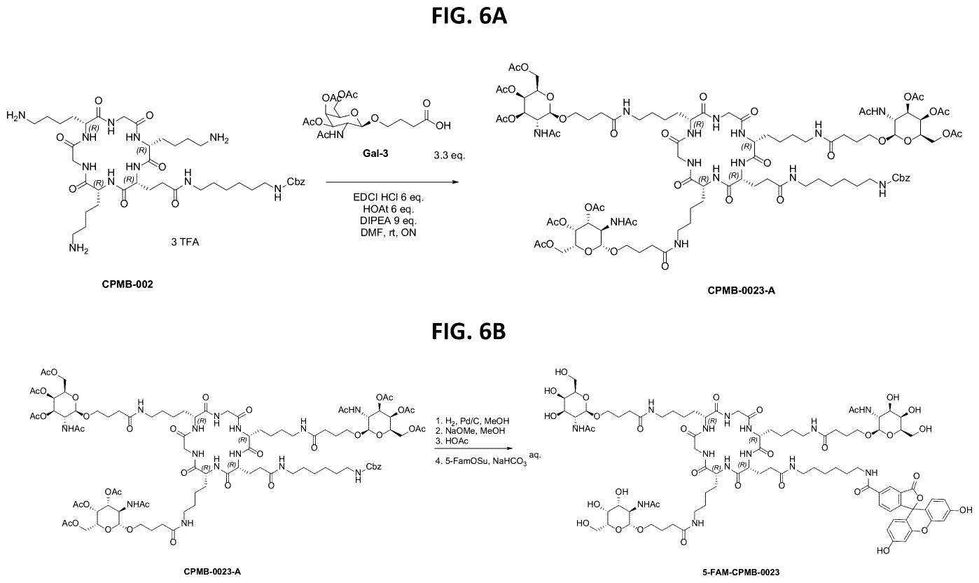

An exemplary synthesis scheme for producing CPMB-0023-A is provided in FIG. 6A.

To a solution of benzyl (6-(3-((2R,5R,11R,17R)-5,11,17-tris(4-aminobutyl)-3,6,9,12,15,18-hexaoxo-1,4,7,10,13,16-hexaazacyclooctadecan-2-yl)propanamido)hexyl) carbamate (CPMB-002) (904 mg, 752 μmol, 1.0 equiv.) in DMF (5 mL) was added EDCI·HCl (865 mg, 4.51 mmol, 6.0 equiv.), HOAt (614 mg, 4.51 mmol, 6.0 equiv.), DIPEA (1.18 mL, 6.76 mmol, 9.0 equiv.), 4-(((2R,3R,4R,5R,6R)-3-acetamido-4,5-diacetoxy-6-(acetoxymethyl)tetrahydro-2H-pyran-2-yl)oxy) butanoic acid (Gal-3) (1.07 g, 2.48 mmol, 3.3 equiv.) in order. The resulting solution was stirred at 25° C. overnight and monitored by LCMS. Upon completion, the solution was diluted with H₂O (15 mL), extracted with DCM (3×20 mL), dried over Na₂SO₄, filtered and concentrated in vacuo. The residue was purified by reversed phase chromatography eluting with H₂O (0.01% v/v TFA)/MeCN (95/5 to 5/95) to afford the compound CPMB-0023-A as white foam (1.49 g, 94% yield).

### Purification Method:

Mobile Phase: A: 0.05% TFA in water; B: 0.05% TFA in acetonitrile
Column: Waters XBridge Prep C18, 19×250 mm, 10 μm, 110 Å
Flow rate: 25 mL/min
Eluants: elution on a lineal density gradient of A/B=71/29-61/39 (20 mins),
fraction at 18.35 min was collected and lyophilized
LCMS: (ESI) m/z=1053.3 [M/2+H]⁺.

### Step 2: Synthesis of 5-Fam-CPMB-0023

An exemplary synthesis scheme for producing 5-Fam-CPMB-0023 is provided in FIG. 6B.

To a solution of CPMB-0023-A (17.0 mg, 8.1 μmol, 1.0 equiv.) in MeOH (2.0 mL) was added Pd/C (4.0 mg, 10.0%). The flask was evacuated and flushed 3 times with H₂. The resulting mixture was stirred for 16 h at 25° C. and monitored by LCMS. Upon completion, the mixture was filtered through a syringe equipped with a filter membrane (0.5 μm, NAVIGATOR®). To the filtrate was added a solution of NaOMe in MeOH (100 μL, 30.0 wt %, 5.4 M). The resulting solution was stirred for 20 min at 25° C. and monitored by LCMS. Upon completion, the solution was neutralized by addition of acetic acid (31.0 μL). The solution was concentrated in vacuo and the residue was dissolved in saturated aqueous NaHCO₃ (1 mL) was diluted with H₂O (1.0 mL), then added 2,5-dioxopyrrolidin-1-yl 3′,6′-dihydroxy-3-oxo-3H-spiro[isobenzofuran-1,9′-xanthene]-5-carboxylate (5.7 mg, 12.1 μmol, 1.5 equiv.). The flask was covered with the aluminum foil to prevent the solution from light. The resulting solution was stirred for 16 h at 25° C. and monitored by LCMS. Upon completion, the solution was diluted with MeCN (2.0 mL) and purified by prep-HPLC to afford 5-Fam-CPMB-0023 as orange solid (8.5 mg, 54% yield).

### Purification Method:

Using TFA buffer: A: 0.05% TFA in water; B: 0.05% TFA in acetonitrile
Column: Phenomenex Gemini C18, 21.2×250 mm, 10 m, 110 Å
Flow rate: 25 mL/min
Eluants: elution on a lineal density gradient of A/B=83/17-73/27 (20 mins), fraction at 17.85 min was collected and lyophilized
LCMS: (ESI) m/z=976.7 [M/2+H]⁺.
HPLC: 98.15% (214 nm), RT=12.866 min
   Mobile Phase: A: Water (0.01% TFA) B: ACN (0.01% TFA)
   Gradient: 5% B for 3 min, increase to 65% B within 20 min, increase to 95% within 2 min, hold for 5 min, back to 5% B within 0.1 min.
   Flow Rate: 1.0 mL/min
   Column: XBridge peptide BEH column C18, 4.6×150 mm, 3.5 μm, 130 Å
   Column Temperature: 20° C.

### Example 14: Synthesis of CPMB-0023

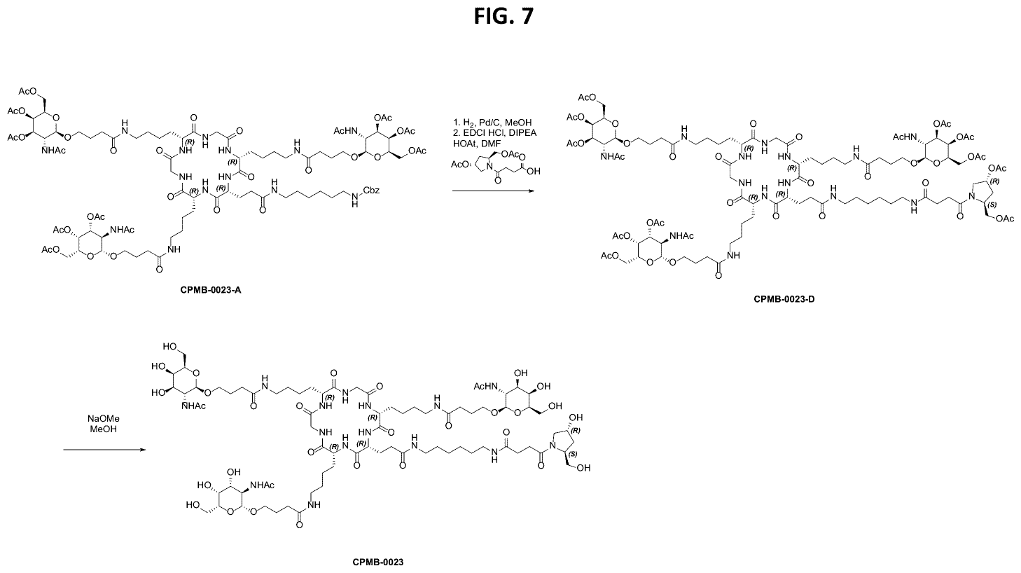

An exemplary synthesis scheme for producing CPMB-0023 is provided in FIG. 7.

### Step 1: Synthesis of CPMB-0023-D

To a solution of CPMB-0023-A (294 mg, 0.14 mmol, 1.0 equiv.) in MeOH (10.0 mL) was added Pd/C (60 mg, 10.0%). The flask was evacuated and flushed 3 times with H₂. The resulting mixture was stirred for 16 h at 25° C. and monitored by LCMS. Upon completion, the mixture was filtered and concentrated. The residue was dissolved in 2 mL DMF and EDCI·HCl (38.8 mg, 0.20 mmol, 1.5 equiv.), HOAt (27.2 mg, 0.20 mmol, 1.5 equiv.), DIPEA (70 μL, 0.41 mmol, 3.0 equiv.), and 4-((2S,4R)-4-acetoxy-2-(acetoxymethyl)pyrrolidin-1-yl)-4-oxobutanoic acid (48.8 mg, 0.16 mmol, 1.2 equiv.) were added in order. The reaction was stirred at rt for 3 h and monitored by LCMS. After the completion of reaction, the mixture was directly purified by reversed phase chromatography eluting with H₂O (0.01% v/v TFA)/MeCN (95/5 to 5/95) to afford the compound CPMB-0023-D as white powder. (303 mg, 96% yield). LCMS: (ESI) m/z=1128.2 [M/2+H]⁺.

### Step 2: Synthesis of CPMB-0023

The compound CPMB-0023-D (303 mg, 0.13 mmol, 1.0 eq.) was dissolved in 5 mL MeOH and a solution of NaOMe in MeOH (30 wt %, 200 μL) was added. The solution was stirred at rt for 20 min and the LCMS indicated that the deacetylation process was completed. The acetic acid (62 μL) was then added to the mixture to neutralize the solution. The mixture was diluted with water (15 mL) and purified with prep-HPLC eluting with H₂O (10 mmol NH₄OAc)/MeCN to afford 98 mg desired compound CPMB-0023 as white foam. (42% yield)

### Purification Method:

Using TFA buffer: A: 0.05% TFA in water; B: 0.05% TFA in acetonitrile
Column: Welch Topsil C18, 21.1×250 mm, 5 μm, 150 Å
Flow rate: 25 mL/min
Eluants: elution on a lineal density gradient of A/B=95/5-85/15 (20 mins), fraction at 20.77 min was collected and lyophilized

LCMS: (ESI) m/z=795.6 [(M-GalNAc)/2+H]⁺.

HPLC: 93.66% (214 nm), RT=14.31 min
Mobile Phase: A: 0.05% TFA in Water B: 0.05% TFA in ACN
Gradient: 2% B for 3 min, increase to 32% B within 20 min, increase to 95% B within 1 min, hold for 6 min, back to 2% B within 0.1 min.
Flow Rate: 1.0 mL/min
Column: XBridge Peptide BEH column C8, 4.6×50 mm, 3.5 m, 130 Å
Column Temperature: 45° C.

### Example 15: Synthesis of CPMB-0023-DMTr

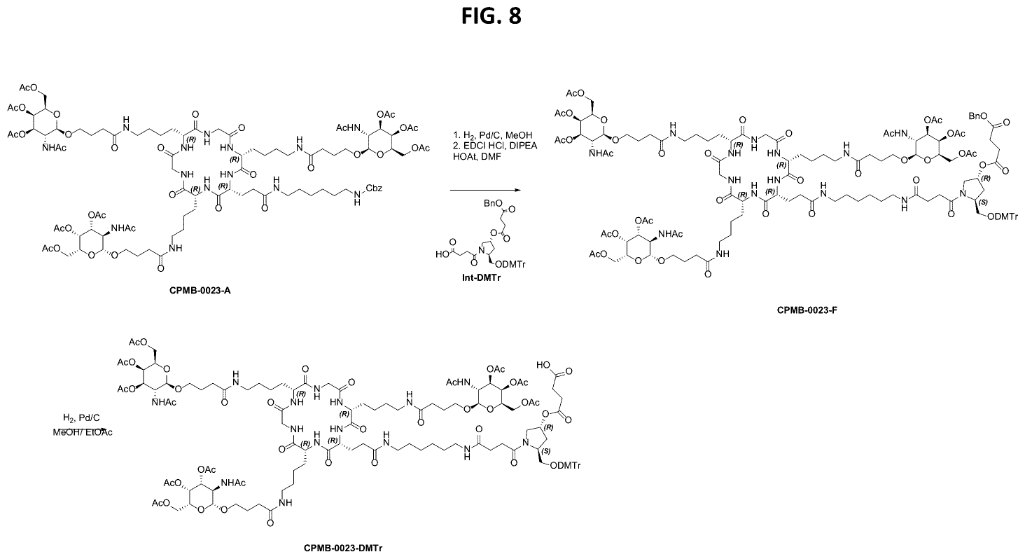

An exemplary synthesis scheme for producing CPMB-0023-DMTr is provided in FIG. 8.

### Step 1: Synthesis of CPMB-0023-F

To a solution of CPMB-0023-A (206 mg, 0.10 mmol, 1.0 equiv.) in MeOH (20.0 mL) was added Pd/C (50 mg, 10.0%). The flask was evacuated and flushed 3 times with H₂. The resulting mixture was stirred for 16 h at 25° C. and monitored by LCMS. Upon completion, the mixture was filtered through a syringe equipped with a filter membrane (0.5 μm, NAVIGATOR©). The residue was dissolved in 1 mL DMF and EDCI·HCl (28.1 mg, 0.15 mmol, 1.5 equiv.), HOAt (20.0 mg, 0.15 mmol, 1.5 equiv.), DIPEA (52 μL, 0.30 mmol, 3.0 equiv.), and Int-DMTr (83.4 mg, 0.12 mmol, 1.2 equiv.) were added in order. The reaction was stirred at rt for 3 h and monitored by LCMS. After the completion of reaction, the mixture was directly purified by reversed phase chromatography eluting with H₂O (0.01% v/v NH₄HCO₃)/MeCN (95/5 to 5/95) to afford the compound CPMB-0023-F as white powder. (114 mg, 44% yield)

### Purification Method:

Mobile Phase: A: 10 mmol NH₄OAc; B: ACN
Column: Waters XBridge Prep C18, 19×250 mm, 10 μm, 130 Å
Flow rate: 25 mL/min
Eluants: elution on a lineal density gradient of A/B=56/44-46/54 (20 mins), fraction at 19.22 min with 100% was collected and lyophilized
LCMS: (ESI) m/z=1181.4 [(M-DMTr)/2+H]⁺.

### Step 2: Synthesis of CPMB-0023-DMTr

To the solution of CPMB-0023-F (114 mg, 0.043 mmol, 1.0 eq.) in MeOH/EtOAc (20.0 mL, 1:1) was added Pd/C (30 mg, 10.0%). The flask was evacuated and flushed 3 times with H₂. The suspension was stirred for 6 h at 25° C. and monitored by LCMS. Upon completion, the solution was filtered and concentrated in vacuo. The residue was purified by prep-HPLC eluting with H₂O (0.01% v/v NH₄HCO₃)/MeCN (95/5 to 5/95) to afford 79 mg desired compound CPMB-0023-DMTr as white powder. (72% yield)

### Purification Method:

Mobile Phase: A: 10 mmol NH₄OAc; B: ACN
Column: Waters XBridge Prep C18, 19×250 mm, 10 μm, 130 Å
Flow rate: 25 mL/min
Eluants: elution on a lineal density gradient of A/B=70/30-60/40 (20 mins), fraction at 19.22 min with 99.1% was collected and lyophilized
LCMS: (ESI) m/z=1285.3 [M/2−H]⁻.
HPLC: >99% (214 nm), RT=10.56 min
Mobile Phase: A: Water (10 mM NH₄HCO₃) B: ACN
Gradient: 5% B for 1 min, increase to 95% B within 20 min, increase to 95% B for 5 min, back to 5% B within 0.1 min.
Flow Rate: 1 mL/min
Column: XBridge Peptide BEH C18, 4.6×150 mm, 3.5 μm, 130 Å
Column Temperature: 45° C.

### Example 16: Synthesis of CPMB-0023-DTMr-PEG4-CPG Resin

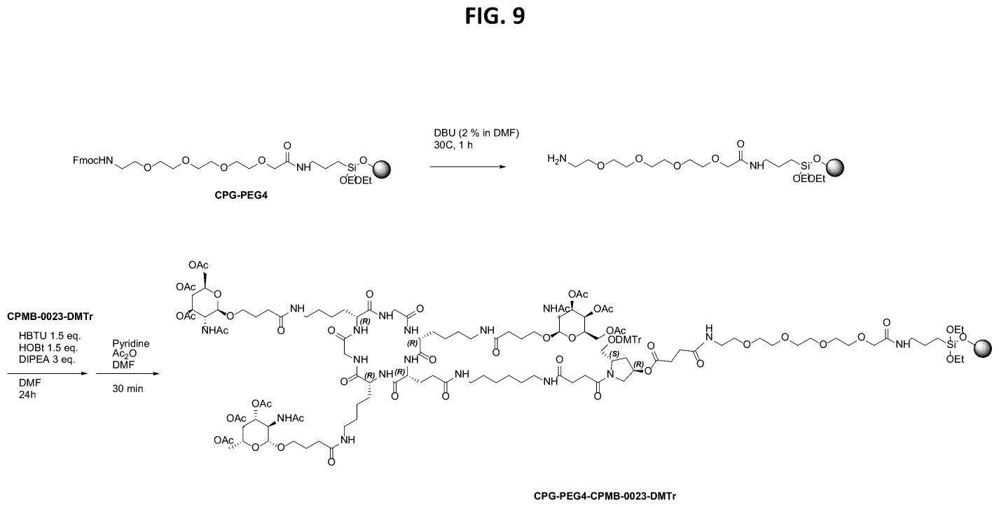

An exemplary synthesis scheme for producing CPMB-0023-DTMr-PEG4-CPG resin is provided in FIG. 9.

Shake the CPG-PEG4 resin (100 mg, 47 μmol/g), in 3 mL DBU (2% in DMF) for 1 h. Kaiser test indicated the completion of de-Fmoc process, then wash the resin with DMF (3 mL×3). Shake the resin with CPMB-0023-DMTr (18 mg, 7 μmol, 1.5 equiv.), DIPEA (2.4 μL, 7 μmol, 3.0 equiv.), HOBt (0.9 mg, 7 μmol, 1.5 equiv.) and HBTU (2.7 mg, 7 μmol, 1.5 equiv.) in DMF (2 mL) at room temperature for 24 h. Then wash the resin with DMF (3 mL×3). Charge the resin with pyridine/Ac₂O (1 mL/20 μL) and shake for 1 h. The resin was then washed with DMF (3 mL×3) and MTBE (5 mL×3) and dried in vacuo. Loading test: take 1.5 mg CPG-PEG4-CPMB-0023-DMTr resin, add 100 μL 1N HCl and 100 μL MeCN, LCMS: S(DMTr⁺)=785.43 mAU, loading=31 μmol/g, 69 mg.

### Example 17: Cellular Uptake of Cyclic Peptide Conjugated with Tri-GalNAc

The human hepatocellular carcinoma (HepG2) cell line was purchased from Shanghai Zhong Qiao Xin Zhou Biotechnology Co., Ltd. and maintained in minimum essential medium (Gibco, ThermoFisher Scientific, USA) with 10% fetal bovine serum (Gibco, ThermoFisher Scientific, USA), 200 units/mL penicillin plus 200 units/mL streptomycin at 37° C. with 5% CO₂.

Cells were seeded in 24-well plates with a density of 1.5×10⁵ cells per well and incubated under 37° C., 5% CO₂. After 18 h of incubation, media were replaced to MEM with 2% FBS and FAM-labeled ligands were added with final concentrations of 1.6, 8, 40 or 200 nM. After 24 h of incubation, cells were washed twice with 1×PBS and analyzed by LSRFortessa (BD Biosciences, NJ, USA). The binding efficiency was evaluated by the proportion of FAM positive cells.

Table 4 below shows the percentage of ligand-bound HepG2 cells. HepG2 cell was treated by 14 cyclic peptide-tri-GalNAc ligand variants with serial diluted concentration of 1.6, 8, 40 or 200 nM. Commercialized tri-GalNAc used in Givosirna was used as positive control and cyclic peptide w/o tri-GalNAc was negative control. Among 14 variants, 5-FAM-CPMB-0013 (ID013), 5-FAM-CPMB-0023 (ID023) and 5-FAM-CPMB-0035 (ID035) had relatively higher binding ability as compared to others and commercialized tri-GalNAc. Equilibrium dissociation constant (kd) for 5-FAM-CPMB-0013 (ID013), 5-FAM-CPMB-0023 (ID023), 5-FAM-CPMB-0035 (ID035) and commercialized tri-GalNAC were 7.0, 8.6, 5.7 and 18 nM, respectively.

TABLE 4
Efficiency of Cellular Uptake of Cyclic
Peptide-Tri-GalNAc Conjugates

| | | 200 | 40 | 8 | 1.6 |
|---|---|---|---|---|---|
| | | nM | nM | nM | nM |
| | Compounds | Efficiency (%) | | | |
| New | 5-FAM-CPMB-0011 (011) | 94.2 | 67.7 | 20.1 | 3.0 |
| design | 5-FAM-CPMB-0012 (012) | 95.9 | 74.2 | 19.6 | 1.2 |
| samples | 5-FAM-CPMB-0013 (013) | 98.6 | 90.7 | 54.6 | 10.5 |
| | 5-FAM-CPMB-0014 (014) | 96.5 | 71.0 | 24.4 | 2.3 |
| | 5-FAM-CPMB-0015 (015) | 99 | 89.6 | 36.3 | 16.3 |
| | 5-FAM-CPMB-0021 (021) | 98.8 | 88.7 | 46.1 | 6.8 |
| | 5-FAM-CPMB-0023 (023) | 99.2 | 91.2 | 47.2 | 6.2 |
| | 5-FAM-CPMB-0025 (025) | 98.2 | 83.1 | 30 | 1.6 |
| | 5-FAM-CPMB-0031 (031) | 98.6 | 87.8 | 44.2 | 6.0 |
| | 5-FAM-CPMB-0033 (033) | 98.2 | 82.9 | 34.5 | 5.3 |
| | 5-FAM-CPMB-0034 (034) | 95.6 | 73.6 | 25.6 | 2.0 |
| | 5-FAM-CPMB-0035 (035) | 99.5 | 95.2 | 63.2 | 11.1 |
| | 5-FAM-CPMB-0311 (0311) | 93.3 | 68.8 | 20.8 | 3.1 |
| | 5-FAM-CPMB-0313 (0313) | 95.1 | 73.2 | 24.6 | 3.0 |
| Positive | Commercialized | 96.2 | 73.2 | 27.3 | 2.5 |
| control | tri-GalNAc | | | | |
| Negative | 5-FAM-CPMB- | 0.4 | 0.2 | 0.1 | 0.1 |
| control | 0031-Ac (031-Ac) | | | | |

### Example 18: Stability Assay of Cyclic Peptide Conjugated with Tri-GalNAc

The cytotoxicity effect of cyclic peptide-tri-GalNAc were evaluated with the viability of HepG2 cells and analyzed by Cell Counting Kit-8 (CCK-8) (Dojindo Laboratories, Japan). The assay was performed according to manufacturer's protocol. Briefly, HepG2 were seeded in 96-well plates with a density of 6×10⁴ cells per well and incubated at 37° C. with 5% CO₂. As cells reached 70% confluence, medium was changed to MEM with 2% FBS and serial diluted cyclic peptide-tri-GalNAc with final concentration of 50, 25, 12.5, 6.25, 3.125 and 1.5625 μM were treated. After 24 h of treatment, 10 μL of CCK-8 solution was added to each well, and incubated for 1˜4 h at 37° C. The absorbance at 450 nm was measured by microplate reader (Thermo scientific, Multiskan sky, MA, USA). The survival ratio of cells treated with ID013, ID023 and commercialized tri-GalNAC are from 97˜100%.

FAM-labeled tri-GalNAc ligands were prepared with sterile water with final concentration of 200 nM. 10 μl of ligands was mixed with 90 μl of human plasma and incubated at 37° C. for 0, 24, 48, 72 h. At the end of incubations, each mixture was added 300 μl of methanol to quench the reaction. The quenched samples were centrifuged at 20,000×g for 5 min and the supernatant was further filtered with 0.45 μm filters (Merck Millipore) to remove debris. The filtered samples were measured by UPLC-FLR detector (Waters, MA, USA).

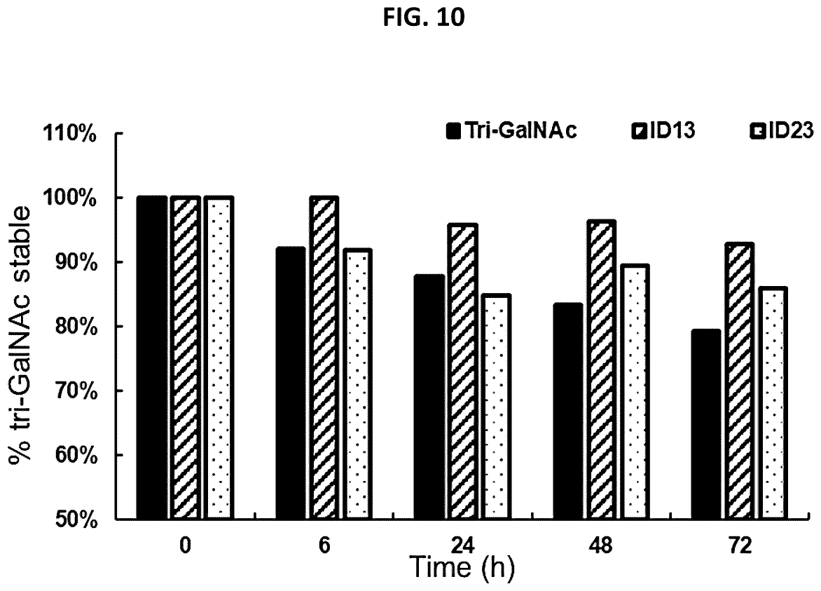

FIG. 10 shows the result of stability assay of 5-FAM-CPMB-0013 (ID013), 5-FAM-CPMB-0023 (ID023) and commercialized tri-GalNAC. 5-FAM-CPMB-0013 (ID013), 5-FAM-CPMB-0023 (ID023) and commercialized tri-GalNAC were incubated with 90% of human plasma for different time points. The amount of each ligand was analyzed and quantified by UPLC system. According to the data, 5-FAM-CPMB-0013 (ID013), 5-FAM-CPMB-0023 (ID023) and commercialized tri-GalNAC are all highly stable within 6 h and then gradually decreased afterward. After 72 h of incubation, remaining amount of 5-FAM-CPMB-0013 (ID013), 5-FAM-CPMB-0023 (ID023) and commercialized tri-GalNAC were 92.7%, 85.8% and 79.1%, respectively.

### Example 19: Endosomal Escape Assay of Cyclic Peptide Conjugated with Tri-GalNAc

HepG2 cells were seeded in 100 mm culture dish with a density of 1×10⁷ cells/10 ml and incubated at 37° C. with 5% C02. After 18 h of incubation, the culture medium was changed to MEM with 2% FBS contained 1 μM final concentration of FAM-labeled ligands. After 3 days of treatment, the endosome and cytosol fractions were separated by endosome isolation and cell fractionation kit (ED-028, Invent Biotechnologies, USA) according to manufacturer's protocol. Briefly, 1.5×10⁵ cells were collected and washed in cold PBS. After centrifugation, the supernatant was removed completely, and the pellet was resuspended with 500 μl of buffer A. Then, the cell suspension was transferred to a filter cartridge, centrifuged at 16,000×g for 30 s. The filtrate and the pellet were mixed thoroughly by 10 s vortex, and centrifuged at 700×g for 3 min to sediment undesired nuclei and intact cells. The supernatant was transferred to a fresh tube and further centrifuged at 16,000×g for 1 h at 4° C. to sediment unwanted larger organelles and plasma membranes in pellet. The supernatant was transferred to a new tube, mixed with buffer B in a ratio of 1:1 and incubated at 4° C. overnight. The mixture was centrifuged at 10,000×g for 30 min at 4° C. The supernatant was the cytosol fraction and the pellet was dissolved in buffer as endosome fraction. The fluorescence intensity of cytosol and endosome fractions were determined by Synergy H1 microplate reader (BioTek, VT, USA) at excitation 490 nm and emission 520 nm.

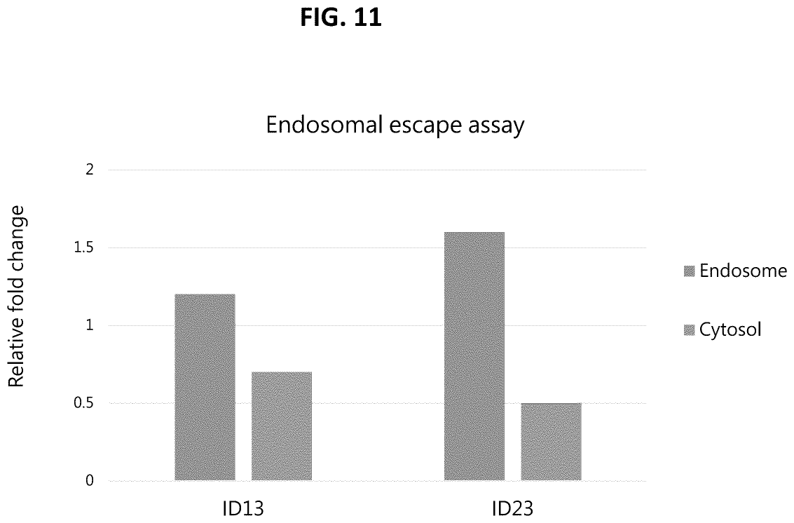

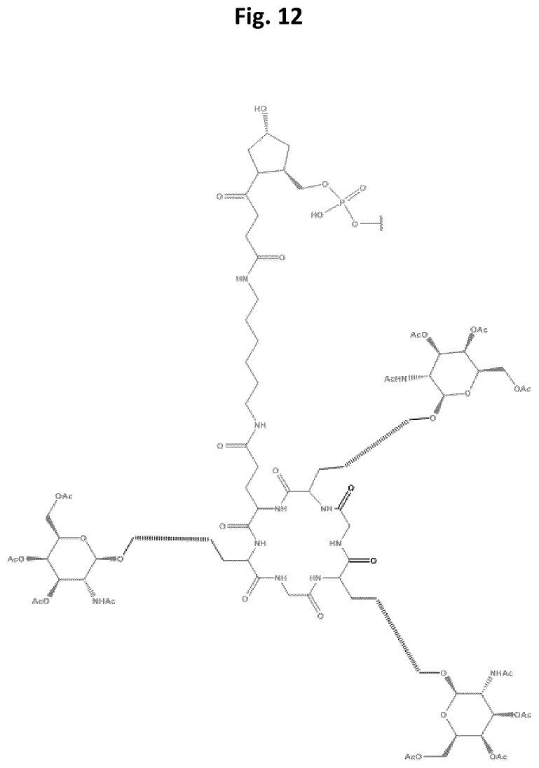

Cells were incubated with 5-FAM-CPMB-0013 (ID013), 5-FAM-CPMB-0023 (ID023), and commercialized tri-GalNAC, and further partitioned into endosome and cytosol fractions. 5-FAM-CPMB-0013 (ID013) and 5-FAM-CPMB-0023 (ID023) were 1.2- and 1.6-fold higher fluorescence intensity than commercialized tri-GalNAC in cytosolic fractions. ID013 and ID023 were 0.7- and 0.5-fold less fluorescence intensity than commercialized tri-GalNAC in endosome fractions, supported that there was more 5-FAM-CPMB-0013 (ID013) or 5-FAM-CPMB-0023 (ID023) released from endosome to cytosol (See FIG. 11). Thus, new developed cyclic peptide-tri-GalNAc have a better capability for endosomal escape.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A conjugate, comprising a cyclic peptide scaffold and one or more N-acetylgalactosamine (GalNAc) moieties,
wherein the cyclic peptide scaffold has 4-10, optionally 4-8, amino acid residues, which comprise Glu, Asp, Lys, Arg, or a combination thereof; and
wherein each of the GalNAc moieties is covalently bound to the cyclic peptide scaffold via a first linker.

2. The conjugate of claim 1, further comprising an agent, wherein the agent is covalently bound to the cyclic peptide scaffold via a second linker.

3. The conjugate of claim 1, wherein the cyclic peptide scaffold has 6 amino acids.

4. The conjugate of claim 1, wherein the cyclic peptide scaffold comprises at least one Glu residue and at least one Lys residue.

5. The conjugate of claim 4, wherein each first linker is covalently bound to the at least one Lys residue.

6. The conjugate of claim 4, wherein the second linker is covalently bound to the at least one Glu residue.

7. The conjugate of claim 1, wherein the cyclic peptide scaffold further comprises Gly, Ala, and/or Val.

8. The conjugate of claim 1, wherein the cyclic peptide scaffold has the amino acid sequence of
(SEQ ID NO: 5)
(a) Lys-Glu-Lys-Gly-Lys-Gly,
or
(SEQ ID NO: 6)
(b) Lys-Glu-Lys-Ala-Lys-Ala.

9. The conjugate of claim 8, wherein one or more amino acid residues in the cyclic peptide scaffold is in D form.

10. The conjugate of claim 1, wherein the cyclic peptide scaffold is selected from the group consisting of CPS-001, CPS-002, CPS-003, and CPS-031, or a functional equivalent thereof; optionally wherein the cyclic peptide scaffold is CPS-001, CPS-002, CPS-003, or CPS-031.

11. The conjugate of claim 1, wherein each first linker comprises a linear chain having 3-8 atoms.

12. The conjugate of claim 11, wherein the 3-8 atoms comprise C, O, or a combination thereof.

13. The conjugate of claim 12, wherein the first linker is the linker in Gal-1, Gal-2, Gal-3, Gal-4, or Gal-5.

14. The conjugate of claim 2, wherein the second linker is a lipid linker, a polyethylene glycol (PEG) linker, or an alkyl amine linker.

15. The conjugate of claim 2, which has the structure of Formula (I): 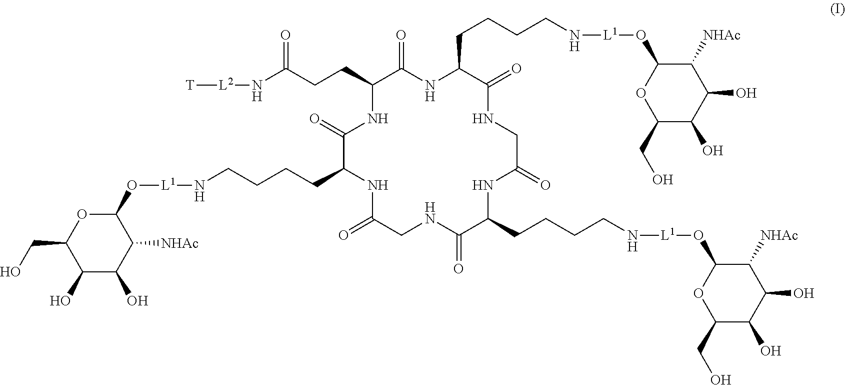
wherein:
T is the agent;
L¹ is the first linker, wherein the first linker is the linker in Gal-1, Gal-2, Gal-3, Gal-4, or Gal-5; and
L² is the second linker.

16. The conjugate of claim 2, which has a structure of Formula (II): 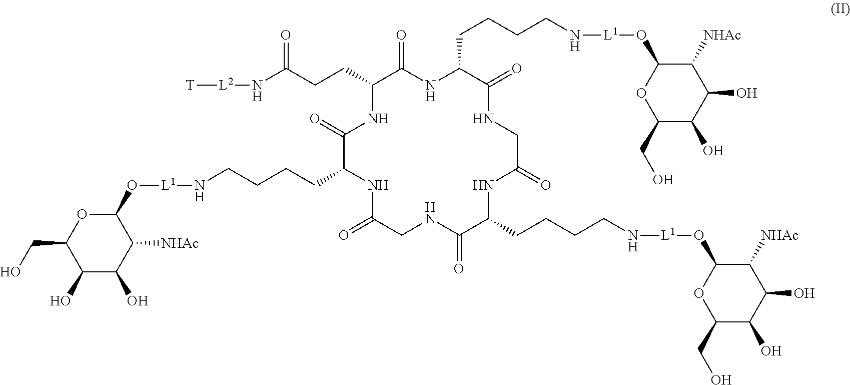
wherein:
T is the agent;
L¹ is the first linker, wherein the first linker is the linker in Gal-1, Gal-2, Gal-3, Gal-4, or Gal-5; and
L² is the second linker.

17. The conjugate of claim 1, wherein the cyclic peptide scaffold has the amino acid sequence of Lys-Glu-Lys-βAla-Lys-βAla (SEQ ID NO: 7).

18. The conjugate of claim 2, which is selected from the group consisting of 5-FAM-CPMB-0011, 5-FAM-CPMB-0012, 5-FAM-CPMB-0013, 5-FAM-CPMB-0014, 5-FAM-CPMB-0015, 5-FAM-CPMB-0021, 5-FAM-CPMB-0023, 5-FAM-CPMB-0025, 5-FAM-CPMB-0031, 5-FAM-CPMB-0033, 5-FAM-CPMB-0034, 5-FAM-CPMB-0035, 5-FAM-CPMB-0311, 5-FAM-CPMB-0313, CPMB-0013, CPMB-0023, CPMB-0013-DOTMr, and CPMB-0023-DOTMr.

19. The conjugate of claim 2, wherein the agent is a diagnostic agent or a therapeutic agent.

20. The conjugate of claim 2, wherein the agent is a small molecule or a nucleic acid.

21. The conjugate of claim 20, wherein the agent is the nucleic acid, which is an siRNA or a nucleic acid aptamer.

22. A pharmaceutical composition, comprising the conjugate of claim 1 and a pharmaceutically acceptable excipient.

23. A method of delivering an agent to liver cells, comprising contacting liver cells with a conjugate of claim 2 or a pharmaceutical composition comprising the conjugate.

24. The method of claim 23, wherein the contacting step comprises administering the conjugate or the composition to a subject in need thereof.

25. The method of claim 23, further comprising administering to a subject in need thereof the liver cells after being contacted with the conjugate or composition.

26. A method for treating a liver disease, comprising administering to a subject in need thereof an effective amount of the conjugate of claim 2 or a pharmaceutical composition comprising the conjugate.


SEQUENCE LISTING (8 sequences)

SEQ ID NO: 1 - protein, 6 aa, Artificial Sequence
  Synthetic
  MISC_FEATURE (2)..(2): modified with Cbz-Linker2
KEKGKG

SEQ ID NO: 2 - protein, 6 aa, Artificial Sequence
  Synthetic
  VARIANT (1), (3), (5): D-Lys
  VARIANT (2)..(2): D-Glu
  MISC_FEATURE (2)..(2): modified with Cbz-Linker2
KEKGKG

SEQ ID NO: 3 - protein, 6 aa, Artificial Sequence
  Synthetic
  VARIANT (2)..(2): D-Glu
  MISC_FEATURE (2)..(2): modified with Cbz-Linker2
KEKGKG

SEQ ID NO: 4 - protein, 6 aa, Artificial Sequence
  Synthetic
  MISC_FEATURE (2)..(2): modified with Cbz-Linker2
  VARIANT (4), (6): B-Ala
KEKAKA

SEQ ID NO: 5 - protein, 6 aa, Artificial Sequence
  Synthetic
KEKGKG

SEQ ID NO: 6 - protein, 6 aa, Artificial Sequence
  Synthetic
KEKAKA

SEQ ID NO: 7 - protein, 6 aa, Artificial Sequence
  Synthetic
  VARIANT (4), (6): B-Ala
KEKAKA

SEQ ID NO: 8 - protein, 6 aa, Artificial Sequence
  Synthetic
  MISC_FEATURE (1), (3), (5): modified with tert-butyloxycarbonyl
  MISC_FEATURE (2)..(2): modified with linker-CBZ
  MISC_FEATURE (6)..(6): modified with CTC resin
KEKGKG